(19) United States
(12) Patent Application Publication
(10) Pub. No.: US 2022/0389074 A1
(43) Pub. Date: Dec. 8, 2022

(54) COMPOSITIONS AND USES THEREOF FOR TREATING, PROGNOSING AND DIAGNOSING PULMONARY HYPERTENSION

(71) Applicant: UNIVERSITY OF PITTSBURGH-OF THE COMMONWEALTH SYSTEM OF HIGHER EDUCATION, Pittsburgh, PA (US)

(72) Inventors: Stephen Yu-Wah CHAN, Pittsburgh, PA (US); Wei SUN, Sewickley, PA (US)

(21) Appl. No.: 17/776,569

(22) PCT Filed: Nov. 12, 2020
(86) PCT No.: PCT/US2020/059969

§ 371 Date: May 12, 2022

Related U.S. Application Data
(60) Provisional application No. 62/934,818, filed on Nov. 13, 2019.

(51) Int. Cl. C07K 14/705; A61P 9/12; A61P 9/10; C12N 15/86

(52) U.S. Cl. CPC C07K 14/705; A61P 9/12; A61P 9/10; C12N 15/86

(57) ABSTRACT

Disclosed herein are compositions and methods for treating pulmonary hypertension whereby a SCUBE1 polynucleotide or polypeptide is administered. Also disclosed herein are methods for diagnosing and/or prognosing pulmonary arterial hypertension or pulmonary hypertension with high pulmonary vascular resistance that include detecting an amount of a SCUBE1 polynucleotide or polypeptide.

### CROSS-REFERENCE TO RELATED APPLICATIONS

This application claims the priority benefit of U.S. Provisional Application No. 62/934,818, filed Nov. 13, 2019, which is incorporated herein by reference in its entirety.

### STATEMENT REGARDING FEDERALLY SPONSORED RESEARCH OR DEVELOPMENT

This invention was made with government support under grant number TR002073 and 2HL129964 awarded by the National Institutes of Health. The government has certain rights in the invention.

### FIELD

The present disclosure relates to compositions and uses thereof for treating pulmonary hypertension.

### BACKGROUND

Pulmonary hypertension (PH) and its particularly severe subtype pulmonary arterial hypertension (PAH) are highly morbid diseases. These conditions are pathologically characterized by progressive pulmonary vascular remodeling and obliteration of pulmonary arterioles, resulting in significantly increased pulmonary vascular resistance (PVR) and pulmonary arterial pressure (PAP) (Simonneau, 2019). The elevated PAP and PVR increase right heart afterload, leading to right ventricular (RV) hypertrophy, dilation, and failure over time (Rabinovitch M, 2012). World Symposium on Pulmonary Hypertension (WSPH) Group 1 PAH is comprised of idiopathic, heritable, and comorbid etiologies, such as connective tissue disorders, infections, and others. As compared to PAH, Group 2PH, i.e., pulmonary venous hypertension (PVH) is considerably more prevalent and develops in the setting of increased pulmonary venous pressure due to left heart failure, yet current targeted therapies are mainly reserved for PAH. However, Group 2 PH is often accompanied by an elevated PVR characteristic of PAH (Simonneau, 2019); under these circumstances, it can be difficult to distinguish between the two classifications without invasive hemodynamic study, and it is unknown if this subgroup shares a common or distinct pathogenesis with PAH.

The onset of PAH symptoms is unfortunately associated with a median survival of only 7 years from the time of diagnosis (Benza R L 2012). PAH is often diagnosed late in the disease course when severe symptoms, such as dyspnea and RV failure, often present (Brown L M 2011). A recent study linking BMP9 plasma levels to portopulmonary hypertension (Nikolic I 2019), a subtype of WSPH Group 1 PAH, indicated the diagnostic utility of utilizing BMP-specific ligands and partners of BMPR2 in this disease. Yet, to date, effective blood or plasma clinical biomarkers that correlate well with early pulmonary vasculature remodeling in PAH or with disease severity have been elusive (Anwar A 2016).

Accordingly, what is needed are compositions and methods for treating and diagnosing pulmonary arterial hypertension. The compositions and methods disclosed herein address these and other needs.

### SUMMARY

Disclosed herein are compositions and methods for treating pulmonary hypertension in a subject comprising administering to the subject a therapeutically effective amount of a SCUBE1 polypeptide or a vector comprising a polynucleotide that encodes SCUBE1 or a functional fragment thereof. The methods can increase angiogenesis, proliferation and survival of pulmonary arterial endothelial cells. Such methods are surprisingly effective at decreasing pulmonary arterial pressure and/or pulmonary vascular resistance in a subject.

Also disclosed herein are methods of diagnosing or prognosing a pulmonary hypertension in a subject comprising detecting a reduction of SCUBE1 in a biological sample derived from the subject relative to a control. In some embodiments, a degree of the reduction positively correlates with severity of the pulmonary arterial hypertension. Such methods are surprisingly effective at diagnosing and prognosing PAH, especially, distinguishing PAH from other cardiopulmonary conditions (e.g., acute bacterial pneumonia, acute lung injury, chronic obstructive pulmonary disease, or ischemic heart disease). In some embodiments, a degree of the reduction positively correlates with severity of the pulmonary vascular resistance. In some embodiments, the methods further comprise administering to the subject a therapeutically effective amount of a SCUBE1 polypeptide or a functional fragment thereof or a vector comprising a polynucleotide that encodes SCUBE1 or a functional fragment thereof.

In some aspects, disclosed herein is a use of a composition for the preparation of a medicament for the treatment of pulmonary hypertension in a subject in need thereof, wherein the medicament comprises a therapeutically effective amount of a SCUBE1 polypeptide or a vector comprising a polynucleotide that encodes SCUBE1 or a functional fragment thereof. In some embodiments, the medicament is for the treatment of pulmonary arterial hypertension. In some embodiments, the medicament is for the treatment of high pulmonary vascular resistance.

### DESCRIPTION OF DRAWINGS

FIGS. 1A-1F show that SCUBE1 is enriched in pulmonary endothelial cells and downregulated by triggering factors driving PAH. FIG. 1A shows abundance of secreted SCUBE1 protein in conditioned media from human PAECs and PASMCs that was determined by immunoblotting (N=3 samples per group). FIGS. 1B-1C show that PAECs were treated with control siRNA (si-NC) or siRNA specific to BMPR2 (si-BMPR2) for 72 hours after which (FIG. 1B) SCUBE1 mRNA expression was measured by RT-qPCR of cellular homogenates (N=3 samples per group) and SCUBE1 protein abundance was quantified by (FIG. 1C) ELISA of conditioned media (N=4 samples per group). FIGS. 1D-1E show that PAECs were stimulated with hypoxia or IL-1β for 48 hours, after which SCUBE1 mRNA (D; N=3 samples per group) and secreted protein (E; N=3 samples per group), were quantified by RT-qPCR and ELISA, respectively. FIG. 1F shows immunoblot of PAEC homogenates (N=3 samples per group) after the above treatments. Immunoblots are representative of three independent experiments with band intensities quantified by densitometry. Data are presented as mean±SD. P-values were calculated by unpaired two-sided t-test, one-way ANOVA with post-hoc Bonferroni test. The comparisons with P>0.05 were not explicitly stated in the panels. AU: arbitrary units.

FIGS. 2A-2D show that HIF-1α expression is induced by both hypoxia and IL-1β and mediates SCUBE1 downregulation. FIG. 2A shows that PAECs were treated with control siRNA (si-NC), siRNA to BMPR2 (si-BMPR2), hypoxia, and IL-1β for 48 hours, and HIF-1α protein expression was determined by immunoblotting (N=3 samples per group). FIG. 2B shows that PAECs were treated with either si-NC or siRNA to HIF-1α (si-HIF1A) and exposed to normoxia, hypoxia, or IL-1β, followed by immunoblotting for SCUBE1 (N=3 samples per group). FIG. 2C shows that, in addition to the above conditions, PAECs were also treated with si-BMPR2, and mRNA expression was determined by RT-qPCR (N=3-6 samples per group). FIG. 2D shows that BMPR2 mRNA expression was quantified in PAECs treated with either si-NC (left bar) or si-HIF1A (right bar) (N=3 samples per group) Immunoblots are representative of three independent experiments with band intensities quantified by densitometry. In FIGS. 2B-2C, open bar is si-NC, closed bar is si-HIF-1α. Data are presented as mean±SD. P-values were calculated by unpaired two-sided t-test, one-way ANOVA with post-hoc Bonferroni test. The comparisons with P>0.05 were not explicitly stated in the panels. AU: arbitrary units.

FIGS. 3A-3K show that SCUBE1 levels modulate endothelial cell pathophenotypes in cultured PAECs. For loss of function analysis, PAECs were treated with SCUBE1 specific siRNAs while control cells were treated with non-specific scrambled control RNAs. FIG. 3A shows that SCUBE1 mRNA levels were determined by RT-qPCR and FIG. 3B shows that secreted SCUBE1 accumulated in cell culture medium was measured with ELISA. The angiogenic potential of cultured PAECs was determined by Matrigel tube formation assay, in which the representative images and quantification with tube joint counts were presented in FIG. 3C. FIG. 3D shows PAEC proliferation and apoptosis determined by BrdU incorporation assay and FIG. 3E shows Caspase 3/7 activity assay. For gain of function analysis, SCUBE1 overexpression in PAECs was achieved through lentiviral transduction of a SCUBE1 transgene. FIG. 3F shows that the transgene efficiency was evidenced by presence of reporter GFP signals in cells infected by blank lentiviral vector control and SCUBE1 overexpression lentiviral vectors. FIG. 3G shows that the overexpression of SCUBE1 mRNA with lentiviral transgene was confirmed with RT-qPCR and FIG. 3H shows that the increased secreted protein accumulated in culture medium from transgenic PAECs was confirmed by ELISA. The enhanced angiogenic potential and proliferation in PAECs infected with SCUBE1 overexpression lentiviral vectors were determined by Matrigel tube formation assay (shown in FIG. 3I) and BrdU incorporation assay (shown in FIG. 3J), respectively. FIG. 3K shows that the reduced apoptosis in PAECs with SCUBE1 overexpression was determined by Caspase 3/7 activity assay. In FIGS. 3A-3E, open bar is si-NC, closed bar is si-SCUBE1. In FIGS. 3G-3K, bar with dots is Lenti-GFP, bar with stripes is Lenti-SCUBE1. The data were derived from 6 independent experiments and presented as mean+SEM. *: p<0.05 with Student t test.

FIGS. 4A-4D show that SMAD1/5/9 phosphorylation connects SCUBE1 expression to BMPR2 signaling. PAECs were treated with SCUBE1 specific siRNAs for SCUBE1 knockdown (non-specific scrambled RNAs as control), or infected with SCUBE1 expression lentiviral vector for SCUBE1 overexpression (blank lentiviral vector with GFP reporter gene as control). FIG. 4A shows BMPR2 mRNA levels determined by RT-qPCR. FIG. 4B shows Western blotting for the phosphorylated (activated) fraction of Smad1/5/9 and Smad2/3, and total Smad1 and Smad2. The densitometry of the blotting was performed to quantify the ratio of phosphorylated Smad1/5/9 to Smad1 (shown in FIG. 4C) and phosphorylated Smad2/3 to Smad2 (shown in FIG. 4D). Open bar is si-NC, closed bar is si-SCUBE1, bar with dots is Lenti-GFP, bar with cross stripes is Lenti-SCUBE1. The data were derived from 3 independent experiments and presented as mean±SEM. *: p<0.05 with Student t test.

FIGS. 5A-5J show that SCUBE1 expression is decreased in rodent models of PAH. Male Sprague-Dawley rats were injected with monocrotaline (MCT) (vs. vehicle (Ctl) or injected with SU5416 (SuHx) and exposed chronically to 10% O₂ for 3 weeks followed by 2 weeks of normoxia (vs. vehicle (Ctl) in normoxia). Male C57BL/6 IL-6 transgenic mice were subjected to 3 weeks of hypoxia (10% O₂) exposure (IL-6/Hx) vs. normoxia (Ctl). Acute bacterial pneumonia (PNA) was generated in male and female C57/BL mice at 0 h (baseline Ctl) and after 48 h following intratracheal administration of K. pneumoniae. Acute myocardial infarction (AMI) was induced by direct ligation of left coronary artery (vs. sham surgery (Ctl)) for 5 days in C57BL/6 mice. FIGS. 5A-5B show data of rat MCT PAH model; FIGS. 5C-5D show data of rat SuHx PAH model; FIGS. 5E-5F show data of mouse IL-6/Hx PAH model; FIGS. 5G-5H show data of mouse PNA model; FIGS. 5I-5J show data of mouse AMI model. SCUBE1 protein levels were determined by ELISA in (FIGS. 5A, 5C, 5E, 5G, 5I) plasma (N=3-7 per group) and (FIGS. 5B, 5D, 5F, 5H, 5J) lung or heart tissue homogenate (N=3-6 per group) collected from euthanized animals Data are presented as mean±SD. P-values were calculated by unpaired two-sided t-test.

FIGS. 6A-6F show that plasma SCUBE1 levels are decreased in WSPH Group 1 PAH patients. FIG. 6A shows that plasma was collected from the pulmonary arteries of patients with WSPH Group 1 (N=62 patients) at the time of right heart catheterization (RHC). Peripheral plasma samples were collected from patients without PH (Non-PH; N=56 patients), patients with COPD (N=39 patients), and ALI (N=39 patients). SCUBE1 protein was quantified and compared across cohorts. FIG. 6B shows that SCUBE1 protein was quantified in lung tissue obtained from rapid autopsy or lung transplant of individuals with WSPH Group 1 PAH (N=8 patients), without PH (Non-PH; N=11 patients), or COPD (N=20 patients). FIG. 6C shows that plasma was collected from Group 2 PH (Group 2 PH, N=16 patients), and SCUBE1 protein levels were compared to Group 1 PAH plasma samples. FIG. 6D shows that SCUBE1 protein was quantified in serum samples collected from patients with coronary angiogram confirmed CAD (N=22) and non-CAD controls (N=21). FIG. 6E shows that SCUBE1 protein was quantified in myocardium tissue homogenates obtained from rapid autopsy or heart transplant of non-diseased individuals (Ctl, N=12), patients with NICM (N=12 patients) and ICM (N=12 patients). FIG. 6F shows ROC curve for sensitivity and specificity analysis between PAH vs. a combined non-PAH cohort composed of control, COPD, and ALI patients (Clopper-Pearson method). Grouped data are presented as median with Q1-Q3 interquartile range. P-values were calculated by Mann-Whitney nonparametric tests for pairwise comparisons, and Kruskal-Wallis test with post-hoc Dunn's Multiple Comparison test. The comparisons with P>0.05 were not explicitly stated in the panels.

FIGS. 7A-7G show that plasma SCUBE1 levels are inversely correlated with hemodynamic markers of disease severity in WSPH Group 1 PAH patients. Plasma SCUBE1 concentration was compared across WSPH Group 1 PAH patients with overall mean PAP (mPAP) and calculated PVR with Spearman correlation (FIGS. 7A and 7C) or trend of change analysis in quartiles (binned based on minimum, 25th percentile, median, 75th percentile and maximum) of mPAP (1st: 15-35 mmHg, 2nd: 36-44 mmHg, 3rd: 45-51 mmHg, 4th: 52-86 mmHg) (FIG. 7B), or PVR (1st: 1.30-4.06 WU, 2nd: 4.07-5.31 WU, 3rd: 5.32-8.42 WU, 4th: 8.42-20.0 WU) (FIG. 7D). FIGS. 7E-7G show that transthoracic echocardiography (TTE) images from the patient cohort managed at UPMC with WSPH Group 1 PAH (N=49 patients) were reviewed, and RV dimensions and TAPSE were directly measured from original TTE images. Plasma SCUBE1 levels were compared within the population based on the degree of RV hypertrophy (FIG. 7E), RV dilation (FIG. 7F), or TAPSE tertile (binned based on minimum, 33th percentile, 66th percentile and maximum. 1st: 2.3-2.8 cm, 2nd: 1.7-2.3 cm, 3rd: 1.2-1.6 cm) (FIG. 7G). Grouped data are presented as median with Q1-Q3 interquartile range. P-values were calculated by Mann-Whitney nonparametric tests for pairwise comparisons, Kruskal-Wallis test for the change across quartiles and tertiles, and Spearman correlation analysis (rho: correlation coefficient).

FIGS. 8A and 8B show efficacy of siRNA knockdown for BMPR2 and HIF-1α genes. Cultured PAECs were incubated with siRNA targeting BMPR2 or HIF-1α, and non-specific scrambled RNAs were used as control. BMPR2 (FIG. 8A) or HIF-1α (FIG. 8B) expression in mRNA levels were determined by RT-qPCR. The data were derived from 3 independent experiments, Data are presented as mean+SD. P-values were calculated by unpaired two-sided t-test. AU: arbitrary units.

FIGS. 9A-9B show a correlation analysis of plasma SCUBE1 with left and right heart filling pressure and cardiac output in WSPH Group 1 PAH patients. Plasma was collected from patients with WSPH Group 1 at the time of right heart catheterization (RHC). Plasma SCUBE1 levels were measured by ELISA. From 62 Group 1 PAH patients where these specific catheterization indices were available, no significant correlation was found between plasma SCUBE 1 levels with pulmonary capillary wedge pressure (PWP; FIG. 9A) and with cardiac index (CI, FIG. 9B). P-values were calculated from Spearman correlation (rho: correlation coefficient).

FIGS. 10A-10I show the expression profile of major endothelial function regulating genes in PAECs treated with hypoxia or IL-1β exposure. Cultured PAECs were treated with hypoxia or IL-1β for 48 hours and mRNA (N=6 samples per group) were quantified by RT-qPCR. The relative change in mRNA expression of angiogenesis, proliferation and apoptosis-related genes VEGF, NOS3, ANG, ANGPT1 (FIGS. 10A-10D), adhesion molecule genes vWF and VECAM1 (FIGS. 10E-10F), and endothelial metabolism related genes PDK1, LDHA, CPT1 (FIGS. 10G-10I) were profiled. Data are presented as mean±SD. P-values were calculated by one-way ANOVA with post-hoc Bonferroni test. The comparisons with P>0.05 were not explicitly stated in the panels.

FIGS. 11A-11D show that plasma SCUBE1 levels are inversely correlated with hemodynamic markers of pulmonary vascular remodeling in WSPH Group 2 PVH patients. Plasma SCUBE1 concentration was compared across WSPH Group 2 PVH patients with overall mean PAP (mPAP, FIG. 11A) and calculated PVR (FIG. 11B) with Spearman correlation. No significant correlation was found between plasma SCUBE 1 levels with pulmonary capillary wedge pressure (PCWP; FIG. 11C) and with cardiac index (CI, FIG. 11D). Plasma was collected from patients with WSPH Group 2 patients (N=14) at the time of right heart catheterization (RHC). Plasma SCUBE1 levels were measured by ELISA. P-values were calculated from Spearman correlation (rho: correlation coefficient).

FIG. 12 is a schematic showing SCUBE1 as a secreted factor downregulated by multiple triggers of PAH, leading to control of BMPR2-specific endothelial pathophenotypes relevant to PAH. Evidences generated from RNA sequencing analysis in BMPR2 mutant cells and biological studies in pulmonary arterial endothelial cells (PAECs) suggest that acquired triggers of pulmonary arterial hypertension (PAH), hypoxia and IL-1β upregulate HIF-1α and consequently downregulate SCUBE1 in PAECs. Deficiency of BMPR2, either from genetic or acquired triggers, also downregulates SCUBE1. Decreased SCUBE1 modulates SMAD1/5/9 signaling downstream of BMPR2, thereby altering PAEC survival, proliferation, and angiogenic potential and leading to pulmonary vascular remodeling, PAH occurrence, and subsequent right heart failure. Decreased plasma SCUBE1 in PAH animal models and patients correlates with indices of PAH, supporting its potential as a clinical marker of disease.

### DETAILED DESCRIPTION

Disclosed herein are compositions and methods of treating a pulmonary hypertension in a subject comprising administering to the subject a therapeutically effective amount of a vector, wherein the vector comprises a polynucleotide that encodes SCUBE1 or a functional fragment thereof. In other aspects, disclosed herein are methods of treating a pulmonary hypertension in a subject comprising administering to the subject a therapeutically amount of a polypeptide, wherein the polypeptide comprises SCUBE1 or a functional fragment thereof. In one example, the subject has a pulmonary arterial hypertension (PAH) prior to treatment. In one example, the subject has a pulmonary vascular hypertension (PVH) with a high pulmonary vascular resistance prior to treatment. Administering the vectors and/or the polypeptides surprisingly mitigates pulmonary arterial hypertension, increases pulmonary arterial endothelial cell angiogenesis, decreases pulmonary arterial pressure, and/or decreases pulmonary vascular resistance in a subject receiving the treatment.

Also disclosed herein are methods of diagnosing, prognosing, and monitoring severity of a pulmonary hypertension in a subject comprising detecting a reduction of SCUBE1 in a biological sample derived from the subject relative to a control. The levels of SCUBE1 in the biological samples surprisingly correlate with the severity of the disorder and can distinguish pulmonary arterial hypertension from pulmonary venous hypertension.

Terms used throughout this application are to be construed with ordinary and typical meaning to those of ordinary skill in the art. However, Applicants desire that the following terms be given the particular definition as provided below.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

The term “angiogenesis” refers to the process by which new blood vessels develop from preexisting vasculature, e.g., capillaries, see e.g., Folkman et al., Nature Med. (1992) 1: 27-21. Angiogenesis is a complex process (see Folkman et al., J Biol Chem. (1992) 267: 10931-4 and Fan et al., Trends Pharmacol Sci. (1995) 16: 57-66; these references and the references cited therein are incorporated herein by reference) that can involve endothelial cell and pericyte activation; basal lamina degradation; migration and proliferation (i.e., cell division) of endothelial cells and pericytes; formation of a new capillary vessel lumen; appearance of pericytes around the new vessels; development of a new basal lamina; capillary loop formation; persistence of involution, differentiation of the new vessels; and, capillary network formation and, eventually, organization into larger microvessels. See, e.g., Safi, J., et al., Mol. Cell Cardiol. (1997) 29: 2311-2325. Compositions can be screened for angiogenic activity in vitro or in vivo. An exemplary in vitro capillary formation assessment uses endothelial cells imbedded in Matrigel matrix (Collaborative Research, Bedford, Mass.), as described by, e.g., Deramaudt, et al., J. Cell. Biochem. (1998) 68: 121-127.

The term “biological sample” as used herein means a sample of biological tissue or fluid. Such samples include, but are not limited to, tissue isolated from animals Biological samples can also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from patient tissues. A biological sample can be provided by removing a sample of cells from an animal, but can also be accomplished by using previously isolated cells (e.g., isolated by another person, at another time, and/or for another purpose), or by performing the methods as disclosed herein in vivo. Archival tissues, such as those having treatment or outcome history can also be used.

A “control” is an alternative subject or sample used in an experiment for comparison purpose. A control can be “positive” or “negative.” In some embodiments, the term “control” refers to a level of a SCUBE1 polypeptide or a SCUBE1 polynucleotide in a sample derived from a pulmonary arterial hypertension free or healthy individual, a sample taken at a different stage in disease development, or a sample from a general or study population.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding trace elements of other ingredients. Embodiments defined by each of these transition terms are within the scope of this invention.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a bacterium, a vector, polynucleotide, cells, salts, esters, amides, proagents, active metabolites, isomers, fragments, analogs, and the like. When the terms “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc. In some aspects, the composition disclosed herein comprises a vector, wherein the vector comprises a polynucleotide that encodes SCUBE1 or a functional fragment thereof. In other aspects, the composition comprises a SCUBE1 polypeptide.

“Effective amount” encompasses, without limitation, an amount that can ameliorate, reverse, mitigate, prevent, or diagnose a symptom or sign of a medical condition or disorder (e.g., pulmonary arterial hypertension). Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter. The term “effective amount of a vector” refers to an amount of a vector sufficient to cause some mitigation of a pulmonary arterial hypertension, and/or related symptoms.

As used herein, “endothelial cell” means a cell which lines the blood and lymphatic vessels. In some embodiments, the endothelial cell is an arterial endothelial cell. In some embodiments, the arterial endothelial cell is a pulmonary arterial endothelial cell.

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as ameliorating pulmonary arterial hypertension.

The term “high pulmonary vascular resistance” is defined herein as resistance equal to or greater than about 2.2 Wood Units (for example, equal to or greater than about 2.2. Wood Units, equal to or greater than about 2.3 Wood Units, equal to or greater than about 2.4 Wood Units, equal to or greater than about 2.5 Wood Units, equal to or greater than about 2.6 Wood Units, equal to or greater than about 2.7 Wood Units, equal to or greater than about 2.8 Wood Units, equal to or greater than about 2.9 Wood Units, equal to or greater than about 3.0 Wood Units, equal to or greater than about 3.1 Wood Units, equal to or greater than about 3.2 Wood Units, equal to or greater than about 3.3 Wood Units, equal to or greater than about 3.4 Wood Units, equal to or greater than about 3.5 Wood Units, equal to or greater than about 3.6 Wood Units, equal to or greater than about 3.7 Wood Units, equal to or greater than about 3.8 Wood Units, equal to or greater than about 3.9 Wood Units, or equal to or greater than about 4.0 Wood Units. In some embodiments, the high pulmonary vascular resistance is equal to or greater than 3.0 Wood Units. In some embodiments, the high pulmonary vascular resistance is equal to or greater than 2.2 Woods Units.

The term “hypertension” is also referred to as “HTN” or “high blood pressure” or and means a medical condition in which the blood pressure in is elevated as compared to a control. This requires the heart to work harder than normal to circulate blood through the blood vessels. Blood pressure is summarized by two measurements, systolic and diastolic, which depend on whether the heart muscle is contracting (systole) or relaxed between beats (diastole) and equate to a maximum and minimum pressure, respectively. Normal blood pressure at rest is within the range of 100-140 mmHg systolic (top reading) and 60-90 mmHg diastolic (bottom reading). High blood pressure is said to be present if it is persistently at or above 140/90 mmHg Hypertension is classified as either primary (essential) hypertension or secondary hypertension; about 90-95% of cases are categorized as “primary hypertension” which means high blood pressure with no obvious underlying medical cause. The remaining 5-10% of cases (secondary hypertension) are caused by other conditions that affect the kidneys, arteries, heart or endocrine system. Hypertension is a major risk factor for stroke, myocardial infarction (heart attacks), heart failure or chronic heart failure (CHF), aneurysms of the arteries (e.g. aortic aneurysm), peripheral arterial disease and is a cause of chronic kidney disease. Even moderate elevation of arterial blood pressure is associated with a shortened life expectancy.

The term “increased” or “increase” as used herein generally means an increase by a statically significant amount; for the avoidance of any doubt, “increased” means an increase of at least 10% as compared to a reference level or a control, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level or a control, or at least about a 2-fold, or at least about a 3-fold, or at least about a 4-fold, or at least about a 5-fold or at least about a 10-fold increase, or any increase between 2-fold and 10-fold or greater as compared to a reference level or a control.

The term “reduced”, “reduce”, “reduction”, or “decrease” as used herein generally means a decrease by a statistically significant amount. However, for avoidance of doubt, “reduced” means a decrease by at least 10% as compared to a reference level or a control, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference or control sample), or any decrease between 10-100% as compared to a reference level or a control.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, Pa., 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, N.J.), polyethylene glycol (PEG), and PLURONICS™ (BASF; Florham Park, N.J.). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “pulmonary hypertension” or “PH” refers herein to an elevation in the pressure in the blood vessels of the lungs as compared to a control. Since the 1st World Symposium on Pulmonary Hypertension (WSPH) organized by the WHO in Geneva in 1973, PH has been grouped into 2 groups: Group 1 (also referred herein as “WSPH Group 1” or “WHO Group 1”) PH (primarily precapillary PH) and Group 2 (also referred herein as “WSPH Group 2” or “WHO Group 2”) PH (primarily postcapillary PH). PH is further subdivided into 5 groups: 1) pulmonary arterial hypertension, 2) pulmonary hypertension due to left heart disease, 3) pulmonary hypertension due to lung disease, 4) pulmonary hypertension due to chronic blood clots, and 5) pulmonary hypertension due to miscellaneous diseases. Accordingly, in some embodiments, a decrease or a reduction of the pulmonary arterial hypertension is a decrease or reduction in the pressure in the blood vessels of the lungs as compared to a control.

As used herein, the term “pulmonary arterial hypertension” or “PAH” refers to an elevation in the pressure in the arteries or arterioles (“precapillaries”) of the lungs as compared to a control. In some embodiments, PAH is associated with malfunction of endothelial cells. In some embodiments, PAH includes pulmonary artery remodeling and/or pulmonary precapillary narrowing. In some embodiments, an elevation in the pressure in the arteries or arterioles is defined as mean pulmonary artery pressure >20 mm Hg, pulmonary vascular resistance greater than 3 Wood Units, and pulmonary capillary wedge pressure greater than 15 mm Hg, as determined by, for example, right heart catheterization hemodynamic assessment. In some embodiments, “PAH” further means that a primary contribution from left heart disease, lung disease, and/or chronic thromboembolic disease has been ruled out. The term “pulmonary arterial hypertension” or “PAH” is intended to include idiopathic PAH, familial PAH, pulmonary veno-occlusive disease (PVOD), pulmonary capillary hemangiomatosis (PCH), persistent pulmonary hypertension of the newborn, or PAH associated with another disease or condition, such as, but not limited to, collagen vascular disease, congenital systemic-to-pulmonary shunts (including Eisenmenger's syndrome), portal hypertension, HIV infection, drugs and toxins, thyroid disorders, glycogen storage disease, Gaucher disease, hereditary hemorrhagic telangiectasia, hemoglobinopathies, myeloproliferative disorders, or splenectomy. A subject suspected of having or having PAH can have or have had a family history of PAH and/or known or suspected genetic predisposition to PAH, exposure to one of the above predisposing factors to PAH, one or more of breathlessness, fatigue, dizziness, echocardiogram indicating PH, and right-heart catheterization indicating PH, increased pulmonary vascular resistance, increased pulmonary pressure, decreased BMPR2 expression/function/signaling, decreased pulmonary arterial endothelial cell angiogenesis, altered endothelial survival, increased vascular cell DNA damage, increased pulmonary vascular inflammation, increased pulmonary vascular stiffening and extracellular matrix remodeling, increased pulmonary artery smooth muscle proliferation, altered vascular cell metabolism, right ventricle (RV) hypertrophy, RV dilation, RV dysfunction, and decreased tricuspid annular plane systolic excursion (TAPSE).

The term “pulmonary venous hypertension” or “PVH” refers to an elevation in the pressure in the veins of the lungs often due to left heart disease and elevated left atrial pressure, but that may also include a reactive precapillary and endothelial component that mimics the hemodynamics of PAH described above. Misdiagnosis and treatment of patients with PVH as instead having PAH can lead to exacerbation of heart failure. It should be understood that included herein are treatments of a subject with PVH, or any PH, having a precapillary and endothelial cell malfunction component as described above in the definition of “PAH.”

“Recombinant” used in reference to a gene refers herein to a sequence of nucleic acids that are not naturally occurring in the genome of the bacterium. The non-naturally occurring sequence may include a recombination, substitution, deletion, or addition of one or more bases with respect to the nucleic acid sequence originally present in the natural genome of the bacterium.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

“Therapeutically effective amount” refers to the amount of a composition such as a vector comprising a polynucleotide that encodes SCUBE1 or a SCUBE1 polypeptide that will elicit the biological or medical response of a tissue, system, animal, or human that is being sought by the researcher, veterinarian, medical doctor or other clinician over a generalized period of time. In some embodiments, a desired response is mitigation, reduction or decrease of a pulmonary arterial hypertension as determined by one or more of right-heart catheterization, echocardiogram, cardiac MRI, electrocardiogram, chest x-ray, a pulmonary function test, an exercise tolerance test, and a blood test that evaluates oxygen levels in the blood or level of right heart strain/function, decreased uptake of fluorodeoxyglucose (FDG) by the right ventricle or pulmonary vessels by PET scan, improved hospitalizations and survival, and/or improvement of symptoms according to NYHA/WSPH functional class. In other embodiments, a desired response is an increase in BMPR2 expression/function/signaling via SMAD apparatus or other downstream mediators, increase in angiogenesis in pulmonary arterial endothelial cells, increased endothelial survival, increased vascular cell DNA damage, increased pulmonary vascular inflammation, increased pulmonary vascular stiffening and extracellular matrix remodeling, increased pulmonary artery smooth muscle proliferation, altered vascular cell metabolism. In some embodiments, a desired response is a decrease of pulmonary arterial pressure, a decrease of pulmonary vascular resistance, and/or an increased survival of a subject having pulmonary arterial hypertension. In some instances, a desired biological or medical response is achieved following administration of multiple dosages of the composition to the subject over a period of days, weeks, or years. The therapeutically effective amount will vary depending on the composition, the disorder or conditions and its severity, the route of administration, time of administration, rate of excretion, drug combination, judgment of the treating physician, dosage form, and the age, weight, general health, sex and/or diet of the subject to be treated. The therapeutically effective amount of a vector comprising a polynucleotide that encodes SCUBE1, a SCUBE1 polypeptide, or a functional fragment thereof as described herein can be determined by one of ordinary skill in the art.

A therapeutically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, such as decreased levels of SCUBE1 in blood and/or a lung tissue, as well as parameters related to a clinically accepted scale of symptoms or markers for a disease or disorder (e.g., pulmonary arterial pressure, and pulmonary vascular resistance). It will be understood, that the total daily usage of the compositions and formulations as disclosed herein will be decided by the attending physician within the scope of sound medical judgment. The exact amount required will vary depending on factors such as the type of disease being treated.

The term “tissue” refers to a group or layer of similarly specialized cells which together perform certain specialized functions. The term “tissue” is intended to include, blood, blood preparations such as plasma and serum, bones, joints, muscles, smooth muscles, lung tissues, and organs.

The terms “treat,” “treating,” “treatment,” and grammatical variations thereof as used herein, include partially or completely delaying, alleviating, mitigating or reducing the intensity of one or more attendant symptoms of a PAH disorder or condition and/or alleviating, mitigating or impeding one or more causes of a PAH disorder or condition. Treatments according to the invention may be applied preventively, prophylactically, pallatively or remedially. Prophylactic treatments are administered to a subject prior to onset (e.g., before obvious signs of a pulmonary arterial hypertension), during early onset (e.g., upon initial signs and symptoms of a pulmonary arterial hypertension), after an established development of a pulmonary arterial hypertension, or at the stage of severe pulmonary arterial hypertension. Prophylactic administration can occur for several minutes to months prior to the manifestation of a pulmonary arterial hypertension.

In some instances, the terms “treat,” “treating,” “treatment,” and grammatical variations thereof, include mitigating a pulmonary arterial hypertension, and/or related symptoms in a subject as compared with prior to treatment of the subject or as compared with incidence of such symptom in a general or study population. Mitigation of a pulmonary arterial hypertension can be determined by one or more of right-heart catheterization indicating decreased pulmonary artery pressure or pulmonary vascular resistance, echocardiogram or cardiac MRI indicating decreased blood pressure in the heart, echocardiogram or cardiac MRI indicating improvement of right ventricular function, dilation, or hypertrophy, chest x-ray indicating no significant further enlargement of right ventricle or pulmonary arteries, an improved pulmonary function test, an improved exercise tolerance test, a blood test that indicates increased oxygen levels in the blood or decreased right heart strain (BNP or pro-NT-BNP), decreased uptake of fluorodeoxyglucose (FDG) by the right ventricle or pulmonary vessels by PET scan, improved hospitalizations and survival, and/or improvement of symptoms according to NYHA/WSPH functional class.

“Vector” used herein means, in respect to a nucleic acid sequence, a nucleic acid sequence comprising a regulatory nucleic acid sequence that controls the replication of an expressible gene. A vector may be either a self-replicating, extrachromosomal vector or a vector which integrates into a host genome. Alternatively, a vector may also be a vehicle comprising the aforementioned nucleic acid sequence. A vector may be a plasmid, bacteriophage, viral particle (isolated, attenuated, recombinant, etc.). A vector may comprise a double-stranded or single-stranded DNA, RNA, or hybrid DNA/RNA sequence comprising double-stranded and/or single-stranded nucleotides. In some embodiments, the vector is a viral vector that comprises a nucleic acid sequence that is a viral packaging sequence responsible for packaging one or a plurality of nucleic acid sequences that encode one or a plurality of polypeptides. In some embodiments, the vector is a plasmid. In some embodiments, the vector is a viral particle. In some embodiments, the vector is viral vector with a natural and/or an engineered capsid. In some embodiments, the viral vector is a lentiviral vector.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g EPV and EBV vectors. Constructs for the recombinant expression of an RNA will generally require regulatory elements, e.g., promoters, enhancers, etc., to ensure the expression of SCUBE1 or a functional fragment thereof in target cells. Other aspects to consider for vectors and constructs are further described below.

Vectors useful for the delivery of SCUBE1 or a functional fragment thereof can include regulatory elements (promoter, enhancer, etc.) sufficient for expression of SCUBE1 or a functional fragment thereof in the desired target cell or tissue. The regulatory elements can be chosen to provide either constitutive or regulated/inducible expression.

### Compositions

Disclosed herein is a method of treating a pulmonary hypertension in a subject comprising administering to the subject a therapeutically effective amount of a vector, wherein the vector comprises a polynucleotide that encodes SCUBE1 or a functional fragment thereof, wherein the administration results in a reduction of a pulmonary arterial hypertension and/or a pulmonary vascular resistance in the subject. In some embodiments, the subject has a pulmonary arterial hypertension (PAH) prior to treatment. In some embodiments, the subject has a pulmonary vascular hypertension (PVH) with a high pulmonary vascular resistance prior to treatment.

In some embodiments, the polynucleotide is a DNA or a RNA. In some embodiments, the vector is a viral vector, such as a lentiviral vector. In some embodiments, the method described herein increases an amount of SCUBE1 in an arterial endothelial cell, such as a pulmonary arterial endothelial cell. In some aspects, disclosed herein is a method of treating a pulmonary hypertension in a subject comprising administering to the subject a therapeutically amount of a polypeptide, wherein the polypeptide comprises SCUBE1 or a functional fragment thereof. In some aspects, administration of the above-mentioned vector and/or the polypeptide increases angiogenesis of the pulmonary arterial endothelial cell, decreases a level of pulmonary arterial pressure in the subject, and/or a decreases a level of pulmonary vascular resistance in the subject. The methods disclosed herein result in a reduction of a pulmonary arterial hypertension and/or a pulmonary vascular resistance in the subject.

Therefore, included herein are compositions that increase an amount of SCUBE1 in or around arterial endothelial cells. Accordingly, disclosed herein are compositions comprising a vector, wherein the vector comprises a polynucleotide that encodes SCUBE1 or a functional fragment thereof. In some embodiments, the polynucleotide is a DNA or a RNA. In some embodiments, the polynucleotide is a DNA. In some embodiments, the polynucleotide is a RNA.

As noted above, the vector can be a nucleic acid sequence comprising a regulatory nucleic acid sequence that controls the replication of an expressible gene. In some embodiments, the vector comprises a promoter operably linked to a second nucleic acid (e.g., polynucleotide encoding a transcription factor), which may include a promoter that is heterologous to the second nucleic acid (e.g., polynucleotide encoding a transcription factor) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). It should be understood herein that the vector of any aspects described herein can further comprise a promoter, an enhancer, an antibiotic resistance gene, and/or an origin, which can be operably linked to one or more of the above noted transcription factors.

In some embodiments, the vector can be a viral vector. “Viral vector” as disclosed herein means, in respect to a vehicle, any virus, virus-like particle, virion, viral particle, or pseudotyped virus that comprises a nucleic acid sequence that directs packaging of a nucleic acid sequence in the virus, virus-like particle, virion, viral particle, or pseudotyped virus. In some embodiments, the virus, virus-like particle, virion, viral particle, or pseudotyped virus is capable of transferring a vector (such as a nucleic acid vector) into and/or between host cells. In some embodiments, the virus, virus-like particle, virion, viral particle, or pseudotyped virus is capable of transferring a vector (such as a nucleic acid vector) into and/or between target cells, such as a hepatocyte in the liver of a subject. Importantly, in some embodiments, the virus, virus-like particle, virion, viral particle, or pseudotyped virus is capable of transporting into cytoplasm and/or a nucleus of a target cell (e.g., a pulmonary arterial endothelial cell). The term “viral vector” is also meant to refer to those forms described more fully in U.S. Patent Application Publication U.S. 2018/0057839, which is incorporated herein by reference for all purposes. In some embodiments, the viral vector is a lentiviral vector.

In some embodiments, the composition comprises one or more viral vectors that contain nucleic acid sequences encoding SCUBE1 or a functional fragment. For example, the composition can comprise a retroviral vector. These retroviral vectors contain the components necessary for the correct packaging of the viral genome and integration into the host cell DNA. More detail about retroviral vectors can be found, for example, in Boesen et al., Biotherapy 6:291-302 (1994). Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993). Lentiviral vectors contemplated for use include, for example, the HIV based vectors described in U.S. Pat. Nos. 6,143,520; 5,665,557; and 5,981,276, which are herein incorporated by reference.

“Signal peptide, CUB domain and EGF like domain containing 1” or “SCUBE1” is a protein that in humans is encoded by the SCUBE1 gene. SCUBE1 belongs to the SCUBE family, which consists of a class of secreted, extracellular proteins that are important in organogenesis. “SCUBE1” refers herein to a polypeptide that, in humans, is encoded by the SCUBE1 gene. In some embodiments, the SCUBE1 polypeptide is that identified in one or more publicly available databases as follows: HGNC: 13441, Entrez Gene: 80274, Ensembl: ENSG00000159307, OMIM: 611746, UniProtKB: Q8IWY4. In some embodiments, the SCUBE polypeptide comprises the sequence of SEQ ID NO: 1, or a polypeptide sequence having at or greater than about 80%, about 85%, about 90%, about 95%, or about 98% homology with SEQ ID NO: 1, or a polypeptide comprising a portion of SEQ ID NO: 1 that is a functional fragment of SCUBE1. The SCUBE1 polypeptide of SEQ ID NO:1 may represent an immature or pre-processed form of mature SCUBE1, and accordingly, included herein are mature or processed portions of the SCUBE1 polypeptide in SEQ ID NO: 1. In some embodiments, a “SCUBE1” used herein may represent a precursor form of the mature SCUBE1, wherein the precursor protein comprises the sequence of SEQ ID NO: 3, or a polypeptide sequence having at or greater than about 80%, about 85%, about 90%, about 95%, or about 98% homology with SEQ ID NO: 3, or a polypeptide comprising a portion of SEQ ID NO: 3 that is a functional fragment of SCUBE1. In some embodiments, the SCUBE1 polynucleotide comprises the sequence of SEQ ID NO: 2, or a polynucleotide sequence having at or greater than about 80%, about 85%, about 90%, about 95%, or about 98% homology with SEQ ID NO: 2, or a polynucleotide comprising a portion of SEQ ID NO: 2.

It is understood that there are numerous amino acid and peptide analogs which can be incorporated into the disclosed compositions Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. D-amino acids, non-natural amino acids, or non-amino acid analogs can be substituted or added to produce a modified protein within the scope of this invention. Cysteine residues can be used to cyclize or attach two or more peptides together. This can be beneficial to constrain peptides into particular conformations. In addition, amino acid side chains of fragments of the protein of the invention can be chemically modified. Another modification is cyclization of the peptide. Accordingly, in order to enhance stability and/or reactivity, a SCUBE1 polypeptide or a functional fragment thereof can be modified to incorporate one or more polymorphisms in the amino acid sequence of the protein resulting from any natural allelic variation.

It is understood herein that the SCUBE1 polypeptide or a functional fragment thereof of any preceding aspect can be operably linked to a homing ligand that specifically binds to a target on a pulmonary arterial endothelial cell. In some embodiments, the ligand is a protein, which can be, for example, L-selectin. Accordingly, in some aspects, the vector comprises a polynucleotide that encodes an L-selectin polypeptide. In some aspects, provided herein is a SCUBE1/L-selectin fusion polypeptide. In some embodiments, the L-selectin polypeptide is that identified in one or more publicly available databases as follows: HGNC: 10720, Entrez Gene: 6402, Ensembl: ENSG00000188404, OMIM: 153240, UniProtKB: P14151. In some embodiments, the L-selectin polypeptide comprises the sequence of SEQ ID NO: 4, or a polypeptide sequence having at or greater than about 80%, about 85%, about 90%, about 95%, or about 98% homology with SEQ ID NO: 4, or a polypeptide comprising a portion of SEQ ID NO: 1 that is a functional fragment of L-selectin. The L-selectin polypeptide of SEQ ID NO:4 may represent an immature or pre-processed form of mature L-selectin, and accordingly, included herein are mature or processed portions of the L-selectin polypeptide in SEQ ID NO: 4.) L-selectin specifically binds to GlyCAM-1 and/or CD34. GlyCAM-1 and CD34 are highly expressed on endothelial cells. In some embodiments, the endothelial cell homing ligand contemplated for use includes, for example, a composition described in U.S. Publication No. 2006/0223756 or U.S. Pat. No. 6,784,153 which are herein incorporated by reference. In some embodiments, the homing ligand is a chemokine receptor that specifically interacts with a chemokine secreted by pulmonary endothelial cells.

In some embodiments, the vector or/and the polypeptide of any preceding aspect is formulated in a pharmaceutically acceptable carrier. In some embodiments, the pharmaceutically acceptable carrier is a microsphere. In some embodiments, the microsphere further comprises the homing ligand noted above. See U.S. Publication No. 2015/0164805 (hereby incorporated by reference) for additional discussion of drug delivery using microspheres.

### Methods of Treatment

The current disclosure demonstrates the surprising finding that increasing the amount of SCUBE1 in a subject having a pulmonary hypertension (e.g., pulmonary arterial hypertension (PAH) or pulmonary vascular hypertension (PVH)) results in increased pulmonary arterial endothelial cell angiogenesis, increased pulmonary arterial endothelial cell proliferation, and/or decreased pulmonary arterial endothelial cell death. It is also shown herein that SCUBE1 affects the activation of a BMPR2/SMAD signaling pathway, and that SCUBE1 is downregulated by genetic and acquired triggers of PAH, including BMPR2 knockdown, hypoxia exposure, and the inflammatory cytokine interleukin-1β treatment.

Therefore, provided herein are methods of treating a pulmonary hypertension in a subject comprising increasing an amount of a SCUBE1 in a subject having pulmonary hypertension (e.g., pulmonary arterial hypertension (PAH) or pulmonary vascular hypertension (PVH)), the increase in SCUBE1 resulting in a reduction or decrease of a pulmonary arterial hypertension and/or a pulmonary vascular resistance in the subject. In some embodiments, the subject has a pulmonary arterial hypertension (PAH) prior to treatment. In some embodiments, the subject has a pulmonary vascular hypertension (PVH) with a high pulmonary vascular resistance prior to treatment.

In some embodiments the amount of a SCUBE1 is increased in a pulmonary artery to a therapeutically effective amount. In some embodiments, the amount of a SCUBE1 is increased in or around an endothelial cell in a pulmonary artery to a therapeutically effective amount. Therefore, included herein is a method of treating a pulmonary hypertension (e.g., pulmonary arterial hypertension (PAH) or pulmonary vascular hypertension (PVH)) comprising administering a composition that increases an amount of SCUBE1 or a functional fragment thereof in an arterial endothelial cell. In some embodiments, the arterial endothelial cell is a pulmonary arterial endothelial cell.

As discussed above, the term “pulmonary arterial hypertension” or “PAH” is intended to include idiopathic PAH, familial PAH, pulmonary veno-occlusive disease (PVOD), pulmonary capillary hemangiomatosis (PCH), persistent pulmonary hypertension of the newborn, or PAH associated with another disease or condition, such as, but not limited to, collagen vascular disease, congenital systemic-to-pulmonary shunts (including Eisenmenger's syndrome), portal hypertension, HIV infection, drugs and toxins, thyroid disorders, glycogen storage disease, Gaucher disease, hereditary hemorrhagic telangiectasia, hemoglobinopathies, myeloproliferative disorders, or splenectomy. Such disorders can result in breathlessness, fatigue, dizziness, echocardiogram indicating PH, and right-heart catheterization indicating PH, increased pulmonary vascular resistance, increased pulmonary pressure, decreased BMPR2 expression/function/signaling, decreased pulmonary arterial endothelial cell angiogenesis, altered endothelial survival, increased vascular cell DNA damage, increased pulmonary vascular inflammation, increased pulmonary vascular stiffening and extracellular matrix remodeling, increased pulmonary artery smooth muscle proliferation, altered vascular cell metabolism, right ventricle (RV) hypertrophy, RV dilation, RV dysfunction, decreased tricuspid annular plane systolic excursion (TAPSE), reduced cardiac output, right heart failure, and/or death. Accordingly, it should be understood that a treatment of PAH may be a treatment of one or more of breathlessness, fatigue, dizziness, echocardiogram indicating PH, and right-heart catheterization indicating PH, increased pulmonary vascular resistance, increased pulmonary pressure, decreased BMPR2 expression/function/signaling, decreased pulmonary arterial endothelial cell angiogenesis, altered endothelial survival, increased vascular cell DNA damage, increased pulmonary vascular inflammation, increased pulmonary vascular stiffening and extracellular matrix remodeling, increased pulmonary artery smooth muscle proliferation, altered vascular cell metabolism, right ventricle (RV) hypertrophy, RV dilation, RV dysfunction, and/or decreased tricuspid annular plane systolic excursion (TAPSE). Treatment can be indicated by one or more of right-heart catheterization indicating decreased pulmonary artery pressure or pulmonary vascular resistance, echocardiogram or cardiac MRI indicating decreased blood pressure in the heart, echocardiogram or cardiac MRI indicating improvement of right ventricular function, dilation, or hypertrophy, chest x-ray indicating no significant further enlargement of right ventricle or pulmonary arteries, an improved pulmonary function test, an improved exercise tolerance test, a blood test that indicates increased oxygen levels in the blood or decreased right heart strain (BNP or pro-NT-BNP), decreased uptake of fluorodeoxyglucose (FDG) by the right ventricle or pulmonary vessels by PET scan, improved hospitalization and survival, and/or improvement of symptoms according to a NYHA/WSPH functional class.

Accordingly, in some embodiments, disclosed herein is a method of treating, preventing, and/or reducing a pulmonary arterial hypertension in a subject in need thereof, comprising administering to the subject a therapeutically effective amount of a vector, wherein the vector comprises a polynucleotide that encodes SCUBE1 or a functional fragment thereof. In some embodiments, disclosed herein is a method of treating, preventing, and/or reducing a pulmonary arterial hypertension in a subject in need thereof, comprising administering to the subject a therapeutically effective amount of a therapeutically amount of a polypeptide, wherein the polypeptide comprises SCUBE1 or a functional fragment thereof. In some embodiments, the administration of the vector and/or the polypeptide increases angiogenesis and proliferation of a pulmonary arterial endothelial cell in the subject. In some embodiments, the administration of the vector and/or the polypeptide decreases cell death of a pulmonary arterial endothelial cell in the subject. In some embodiments, the administration of the vector and/or the polypeptide decreases a level of pulmonary arterial pressure in the subject. In some embodiments, the administration of the vector and/or the polypeptide decreases a level of pulmonary vascular resistance in the subject. In some embodiments, the administration of the vector and/or the polypeptide mitigates one or more of breathlessness, fatigue, dizziness, echocardiogram indicating PH, and right-heart catheterization indicating PH, increased pulmonary vascular resistance, increased pulmonary pressure, decreased BMPR2 expression/function/signaling, decreased pulmonary arterial endothelial cell angiogenesis, altered endothelial survival, increased vascular cell DNA damage, increased pulmonary vascular inflammation, increased pulmonary vascular stiffening and extracellular matrix remodeling, increased pulmonary artery smooth muscle proliferation, altered vascular cell metabolism, right ventricle (RV) hypertrophy, RV dilation, RV dysfunction, and decreased tricuspid annular plane systolic excursion (TAPSE). It should be understood and herein contemplated that the terms “increase” and “decrease” used herein can refer to an increase or decrease as compared to prior to the treatment of the subject or as compared with incidence of such symptom in a general or study population.

In some embodiments, the “high pulmonary vascular resistance” is equal to or greater than about 2.2 Wood Units. In some embodiments, the high pulmonary vascular resistance is equal to or greater than about 3.0 Wood Units. In some embodiments, the the high pulmonary vascular resistance is equal to or greater than about 2.3 Wood Units, 2.4 Wood Units, 2.5 Wood Units, 2.6 Wood Units, 2.7 Wood Units, 2.8 Wood Units, 2.9 Wood Units, 3.0 Wood Units, 3.1 Wood Units, 3.2 Wood Units, 3.3 Wood Units, 3.4 Wood Units, 3.5 Wood Units, 3.6 Wood Units, 3.7 Wood Units, 3.8 Wood Units, 3.9 Wood Units, or 4.0 Wood Units.

BMPR2, or bone morphogenetic protein receptor type 2, and the relevant SMAD signaling may be at the centerpiece of PAH pathogenesis. In some embodiments, BMPR2 is as identified in one or more publicly available databases as follows: HGNC: 1078; Entrez Gene: 659; Ensembl: ENSG00000204217; OMIM: 600799 UniProtKB: Q13873. SCUBE1 is a co-activator of BMPR2. Upon activation, BMPR2 transduces signals from the membrane to nucleus by phosphorylating SMAD transcriptional factors. SCUBE1 deficiency is shown herein to recapitulate phenotypes associated with BMPR2 deficiency, including decreased angiogenic potential, decreased proliferation, and increased apoptosis; while SCUBE1 overexpression displays converse effects and reverses the phenotypes associated with multiple known PAH. Therefore, it should be understood that included herein is a method of administering a vector and/or the polypeptide of any preceding aspect for the correction of BMPR2-relevant SMAD signaling in an endothelial cell (e.g., a pulmonary arterial endothelial cell), resulting in the mitigation of a pulmonary arterial hypertension in a subject.

In some embodiments, the SCUBE1 polypeptide is that identified in one or more publicly available databases as follows: HGNC: 13441, Entrez Gene: 80274, Ensembl: ENSG00000159307, OMIM: 611746, UniProtKB: Q8IWY4. In some embodiments, the SCUBE polypeptide comprises the sequence of SEQ ID NO: 1, or a polypeptide sequence having at or greater than about 80%, about 85%, about 90%, about 95%, or about 98% homology with SEQ ID NO: 1, or a polypeptide comprising a portion of SEQ ID NO: 1 that is a functional fragment of SCUBE1. The SCUBE1 polypeptide of SEQ ID NO:1 may represent an immature or pre-processed form of mature SCUBE1, and accordingly, included herein are mature or processed portions of the SCUBE1 polypeptide in SEQ ID NO: 1. In some embodiments, a “SCUBE1” used herein may represent a precursor form of the mature SCUBE1, wherein the precursor protein comprises the sequence of SEQ ID NO: 3, or a polypeptide sequence having at or greater than about 80%, about 85%, about 90%, about 95%, or about 98% homology with SEQ ID NO: 3, or a polypeptide comprising a portion of SEQ ID NO: 3 that is a functional fragment of SCUBE1. In some embodiments, the SCUBE1 polynucleotide comprises the sequence of SEQ ID NO: 2, or a polynucleotide sequence having at or greater than about 80%, about 85%, about 90%, about 95%, or about 98% homology with SEQ ID NO: 2, or a polynucleotide comprising a portion of SEQ ID NO: 2. In those embodiments in which a therapeutically effective amount of a vector comprising a SCUBE1 polynucleotide, or a fragment thereof, is administered, the polynucleotide can be a DNA or a RNA. In some embodiments, the polynucleotide is a DNA. In some embodiments, the polynucleotide is a RNA. In some embodiments, the vector can be a viral vector. The term “viral vector” is also meant to refer to those forms described more fully in U.S. Publication 2018/0057839, which is incorporated herein by reference for all purposes. In some embodiments, the viral vector is a lentiviral vector.

In another aspect, provided herein is a method of treating a pulmonary hypertension (e.g., pulmonary arterial hypertension (PAH) or pulmonary vascular hypertension (PVH) with a high pulmonary vascular resistance) in a subject suspected of having or having PAH or PVH comprising administering to the subject a therapeutically amount of a polypeptide, wherein the polypeptide comprises SCUBE1 or a functional fragment thereof. The increase in SCUBE1 results in a reduction or decrease of a pulmonary arterial hypertension and/or a pulmonary vascular resistance in the subject. In some embodiments, the subject has a pulmonary arterial hypertension (PAH) prior to treatment. In some embodiments, the subject has a pulmonary vascular hypertension (PVH) with a high pulmonary vascular resistance prior to treatment.

In some embodiments, the SCUBE1 polypeptide comprises the sequence of SEQ ID NO: 1, or a polypeptide sequence having at or greater than about 80%, about 85%, about 90%, about 95%, or about 98% homology with SEQ ID NO: 1, or a polypeptide comprising a portion of SEQ ID NO: 1. The SCUBE1 polypeptide of SEQ ID NO:1 may represent an immature or pre-processed form of mature SCUBE1, and accordingly, included herein are mature or processed portions of the SCUBE1 polypeptide in SEQ ID NO: 1. As noted above, SCUBE1 is a co-activator of BMPR2. Therefore, it is understood herein that the polypeptide disclosed herein can be a fragment of SCUBE1 that forms a protein structure for interacting with BMPR2, resulting an increase of pulmonary arterial endothelial cell angiogenesis and/or mitigation of pulmonary arterial hypertension in the subject.

It is understood that there are numerous amino acid and peptide analogs which can be incorporated into the disclosed compositions Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. D-amino acids, non-natural amino acids, or non-amino acid analogs can be substituted or added to produce a modified protein within the scope of this invention. Cysteine residues can be used to cyclize or attach two or more peptides together. This can be beneficial to constrain peptides into particular conformations. In addition, amino acid side chains of fragments of the protein of the invention can be chemically modified. Another modification is cyclization of the peptide. Accordingly, in order to enhance stability and/or reactivity, SCUBE1 or a functional fragment thereof can be modified to incorporate one or more polymorphisms in the amino acid sequence of the protein resulting from any natural allelic variation.

It is understood herein that SCUBE1 or a functional fragment thereof of any preceding aspect can be operably linked to a homing ligand that specifically binds to a target on a pulmonary arterial endothelial cell. In some embodiments, the ligand is a protein, which can be, for example, L-selectin that binds to GlyCAM-1 and/or CD34. GlyCAM-1 and CD34 are highly expressed on endothelial cells. In some embodiments of the methods, the L-selectin polypeptide is that identified in one or more publicly available databases as follows: HGNC: 10720, Entrez Gene: 6402, Ensembl: ENSG00000188404, OMIM: 153240, UniProtKB: P14151. In some embodiments, the L-selectin polypeptide comprises the sequence of SEQ ID NO: 4, or a polypeptide sequence having at or greater than about 80%, about 85%, about 90%, about 95%, or about 98% homology with SEQ ID NO: 4, or a polypeptide comprising a portion of SEQ ID NO: 1 that is a functional fragment of L-selectin. The L-selectin polypeptide of SEQ ID NO:4 may represent an immature or pre-processed form of mature L-selectin, and accordingly, included herein are mature or processed portions of the L-selectin polypeptide in SEQ ID NO: 4. In some embodiments, the endothelial cell homing ligand is, for example, a composition described in U.S. Publication No. 2006/0223756 or U.S. Pat. No. 6,784,153, which are herein incorporated by reference. In some embodiments, the endothelial cell homing ligand is a chemokine receptor that specifically interacts with a chemokine secreted by pulmonary arterial endothelial cells.

In some embodiments, the vector or/and the polypeptide of any preceding aspect is formulated in a pharmaceutically acceptable carrier. In some embodiments, the pharmaceutically acceptable carrier is a microsphere. In some embodiments, the microsphere further comprises the homing ligand noted above. See U.S. Publication No. 2015/0164805 (hereby incorporated by reference) for additional discussion of drug delivery using microspheres.

The disclosed methods can be performed any time prior to the onset of pulmonary hypertension. In one aspect, the disclosed methods can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years; 12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the onset of pulmonary arterial hypertension; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the onset of pulmonary hypertension. Dosing frequency for the composition of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, two times per day, three times per day, four times per day, five times per day, six times per day, eight times per day, nine times per day, ten times per day, eleven times per day, twelve times per day, once every 12 hours, once every 10 hours, once every 8 hours, once every 6 hours, once every 5 hours, once every 4 hours, once every 3 hours, once every 2 hours, once every hour, once every 40 min, once every 30 min, once every 20 min, or once every 10 min. Administration can also be continuous and adjusted to maintaining a level of the compound within any desired and specified range.

In some embodiments, the method of any preceding aspect further comprises a diagnosis or prognosis of PAH or PVH with high pulmonary vascular resistance based upon a reduction of SCUBE1 in a biological sample derived from the subject relative to a control. The biological sample can be, for example, a blood sample, a serum sample, a plasma sample, a lung tissue sample, and/or a lung fluid sample. Exemplary methods of such prognosis and diagnosis are provided below.

It should be understood that identifying a level of SCUBE1 in a biological sample derived from the subject relative to a control can be done prior to the treatment, during the course of the treatment, and/or after the treatment. Therefore, in one embodiment, the method of treating a pulmonary hypertension (e.g., pulmonary arterial hypertension (PAH) or pulmonary vascular hypertension (PVH)) of any preceding aspect further comprises a step of monitoring and/or assessing the efficacy of the method in the subject, wherein the step comprises identifying a level of SCUBE1 in a biological sample derived from the subject relative to a control prior to the treatment, during the course of the treatment, and/or after the treatment. In these embodiments, an increase in a level of SCUBE1 indicates the efficacy of the method of treatment. These methods allow for monitoring of a SCUBE1 level in a biological sample (e.g., a plasma sample) over an extended period of time, such as years.

### Methods of Diagnosis, Prognosis and Monitoring Efficacy

In some aspects, disclosed herein is a method of diagnosing pulmonary arterial hypertension or pulmonary vascular hypertension with high pulmonary vascular resistance in a subject comprising detecting a reduction of a SCUBE1 polynucleotide or polypeptide in a biological sample derived from the subject relative to a control, and diagnosing the subject with the pulmonary arterial hypertension or pulmonary vascular hypertension with high pulmonary vascular resistance following the detection of the reduction of SCUBE1. It is a surprising finding of the present invention that plasma SCUBE1 levels correlate with mean pulmonary artery pressures and pulmonary vascular resistance. While some candidate biomarkers of PAH have been previously identified, these biomarkers have not been shown to correlate with hemodynamic parameters. Accordingly, the methods disclosed herein are an advancement over the prior art methods that focused on non-specific indicators of right ventricular failure, angiogenesis, or inflammation and did not correlate with hemodynamic parameters. See, e.g., Malhotra R 2013, which showed that plasma levels of soluble endoglin, a co-receptor involved in BMP signaling, are elevated in PAH and predict functional class. The present disclosure further includes methods of distinguishing pulmonary arterial hypertension from other cardiopulmonary conditions (e.g., pulmonary vascular resistance, chronic obstructive pulmonary disease, or ischemic heart disease). In these methods, detection a reduction of SCUBE1 in a biological sample derived from the subject relative to a control indicates pulmonary arterial hypertension and not pulmonary vascular resistance, chronic obstructive pulmonary disease, and/or ischemic heart disease.

It should be understood herein that the “reduction” can be a decrease by at least 10% as compared to a reference level or control, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample or control), or any decrease between 10-100% as compared to a reference level or control. The term “control” used herein refers to a level of a SCUBE1 polypeptide or a SCUBE1 polynucleotide in a sample derived from a pulmonary arterial hypertension free or healthy individual, a sample obtained at a different stage in disease development, or a sample or samples obtained from a general or study population. The biological sample used in the methods of diagnosis or prognosis can be, for example, a blood sample, a serum sample, a plasma sample, a lung tissue sample, and/or a lung fluid sample. In some embodiments, the biological sample is a plasma sample. In some embodiments, the biological sample is a lung tissue sample.

Accordingly, included herein are methods for diagnosing or prognosing pulmonary arterial hypertension or pulmonary vascular hypertension with high pulmonary vascular resistance comprising detecting a reduction in a level of a SCUBE1 polypeptide or a SCUBE1 polynucleotide in a sample derived from a subject having or being suspected of having pulmonary arterial hypertension or pulmonary vascular hypertension with high pulmonary vascular resistance. In some embodiments, the level of the SCUBE1 polypeptide in a subject's plasma sample is less than about 100 ng/ml, 90 ng/ml, 80 ng/ml, 70 ng/ml, 60 ng/ml, 50 ng/ml, 40 ng/ml, 30 ng/ml, 20 ng/ml, 18 ng/ml, 16 ng/ml, 14 ng/ml, 12 ng/ml, 10 ng/ml, 8 ng/ml, 7 ng/ml, 6 ng/ml, 6.5 ng/ml, 5.0 ng/ml, 4.5 ng/ml, 4.0 ng/ml, 3.5 ng/ml, 3.0 ng/ml, 2.5 ng/ml, 2.0 ng/ml, 1.5 ng/ml, 1.0 ng/ml, 0.5 ng/ml, 0.25 ng/ml, 0.1 ng/ml, 0.05 ng/ml, or 0.01 ng/ml. In some embodiments, the level of the SCUBE1 polypeptide in a subject's lung tissue sample is less than about 100 ng/mg, 90 ng/mg, 80 ng/mg, 70 ng/mg, 60 ng/mg, 50 ng/mg, 40 ng/mg, 30 ng/mg, 20 ng/mg, 18 ng/mg, 16 ng/mg, 14 ng/mg, 12 ng/mg, 10 ng/mg, 8 ng/mg, 7 ng/mg, 6 ng/mg, 6.5 ng/mg, 5.0 ng/mg, 4.5 ng/mg, 4.0 ng/mg, 3.5 ng/mg, 3.0 ng/mg, 2.5 ng/mg, 2.0 ng/mg, 1.5 ng/mg, 1.0 ng/mg, 0.5 ng/mg, 0.25 ng/mg, 0.1 ng/mg, 0.05 ng/mg, or 0.01 ng/mg. In some embodiments, a subject having PAH has a plasma or lung tissue level of SCUBE1 between 0.0 ng/ml and 20 ng/ml. In other embodiments, a subject having PAH has a plasma or lung tissue level of SCUBE1 between 0.0 ng/ml and 50 ng/ml. In other embodiments, a subject having PAH has a plasma or lung tissue level of SCUBE1 between 0.0 ng/ml and 100 ng/ml. Levels of SCUBE1 polypeptides can be quantified by an immunodetection method. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (1-RAP/FLAP). In some embodiments, levels of SCUBE1 can be quantified using Mass Spectrometry.

Levels of SCUBE1 polynucleotides can be quantified using PCR, such as real-time PCR. The technique of PCR is described in numerous publications, including, PCR: A Practical Approach, M. J. McPherson, et al., IRL Press (1991), PCR Protocols: A Guide to Methods and Applications, by Innis, et al., Academic Press (1990), and PCR Technology: Principals and Applications for DNA Amplification, H. A. Erlich, Stockton Press (1989). PCR is also described in many U.S. patents, including U.S. Pat. Nos. 4,683,195; 4,683,202; 4,800,159; 4,965,188; 4,889,818; 5,075,216; 5,079,352; 5,104,792; 5,023,171; 5,091,310; and 5,066,584, each of which is incorporated by reference herein.

The present disclosure demonstrates a negative correlation between SCUBE1 levels and severity of pulmonary arterial hypertension, wherein the subject with severe pulmonary arterial hypertension has indices including right ventricle (RV) hypertrophy, moderate to severe RV dilation, or decreased tricuspid annular plane systolic excursion (TAPSE). Therefore, disclosed herein is a method of diagnosing, prognosing, or monitoring the severity of pulmonary arterial hypertension in a subject comprising detecting a reduction of SCUBE1 in a biological sample derived from the subject relative to a control, wherein the reduction of SCUBE1 by about 10% to about 99% relative to the control indicates a pulmonary arterial hypertension. In some embodiments, the reduction of SCUBE1 in a biological sample derived from the subject by about 10% to about 50% relative to the control indicates a mild to moderate PAH, wherein the reduction can be, for example, about 10%, 15%, 20%, 25%, 30%, 35%, 40%, 45%, or 50%. In some embodiments, the reduction of SCUBE1 in a biological sample derived from the subject by more than 50% relative to the control indicates a severe PAH, wherein the reduction can be, for example, more than 50%, more than 55%, more than 60%, more than 65%, more than 70%, more than 75%, more than 80%, more than 85%, more than 90%, more than 95%, or more than 99%. The biological sample can be, for example, a blood sample, a serum sample, a plasma sample, a lung tissue sample, and/or a lung fluid sample. In some embodiments, the biological sample is a plasma sample. In some embodiments, the biological sample is a lung tissue sample. The control used herein refers to a level of a SCUBE1 polypeptide or a SCUBE1 polynucleotide in a pulmonary arterial hypertension free, healthy biological sample, in a sample derived from a pulmonary arterial hypertension free, healthy individual, in sample at different stages in disease development (e.g., an earlier stage of pulmonary hypertension), or a level in a general or study population. In some embodiments, the subject has an increased level of pulmonary arterial pressure relative to a control. In some embodiments, the subject has an increased level of pulmonary vascular resistance relative to a control.

It should be understood that a reduction of a level of SCUBE1 in a biological sample derived from a subject can occur prior to onset of pulmonary arterial hypertension or any of the related symptoms noted above. Indeed, the present disclosure shows that plasma SCUBE1 concentration is independent of cardiac index, indicating its utility to detect PAH before symptoms develop.

In some embodiments, the method of diagnosing, prognosing, and/or monitoring severity of a pulmonary hypertension in a subject of any preceding aspects, further comprises administering to the subject a therapeutically effective amount of a vector, wherein the vector comprises polynucleotide that encodes SCUBE1 or a functional fragment thereof. In some embodiments, the polynucleotide is a DNA or a RNA. In some embodiments, the polynucleotide is a DNA. In some embodiments, the vector is a viral vector. In some embodiments, the viral vector is a viral vector. In some embodiments, the administration of the vector increases angiogenesis of a pulmonary arterial endothelial cell form the subject. In some embodiments, the administration of the vector decreases a level of pulmonary arterial pressure in the subject. In some embodiments, the administration of the vector decreases a level of pulmonary vascular resistance in the subject.

In some embodiments, the method of diagnosing, prognosing, and/or monitoring severity of a pulmonary arterial hypertension in a subject of any preceding aspects, further comprises administering to the subject a therapeutically effector amount of a polypeptide, wherein the polypeptide comprises SCUBE1 or a functional fragment thereof. In some embodiments, the administration of the polypeptide increases angiogenesis of a pulmonary arterial endothelial cell form the subject. In some embodiments, the administration of the polypeptide decreases a level of pulmonary arterial pressure in the subject. In some embodiments, the administration of the polypeptide decreases a level of pulmonary vascular resistance in the subject.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Treatment and Diagnosis of Pulmonary Arterial Hypertension

Despite progress in the era of pulmonary vasodilators, the 5-year mortality in PAH approaches 35-40% for incident and prevalent cases (Farber H W, 2015). The diagnosis of PAH remains challenging given its non-specific clinical symptomatology and the necessity for invasive hemodynamics. Specifically, common acute or chronic pulmonary and cardiovascular diseases independent of PH often present with similar clinical features as PH such as dyspnea or exercise intolerance. Thus, PH can be overlooked, delaying diagnosis on average by 3-4 years and often until severe symptoms and RV failure present (Brown L M, 2011). Clinically, no reliable blood test is available to distinguish suspected PAH from other cardiopulmonary conditions and thus help to convince a clinician to pursue further invasive hemodynamic measurement. A recent study linking BMP9 plasma levels to portopulmonary hypertension (Nikolic I, 2019), a subtype of WSPH Group 1 PAH, indicated the diagnostic utility of utilizing BMP-specific ligands and partners of BMPR2 in this disease. Yet, to date, effective blood or plasma clinical biomarkers that correlate well with early pulmonary vasculature remodeling in PAH or with disease severity have been elusive (Anwar A, 2016).

Multiple subtypes of PH including PAH are driven by pulmonary endothelial cell (EC) dysfunction. It is thought that deficiency of BMPR2, either from genetic or acquired means, drives alterations of downstream signaling, leading to endothelial apoptosis and deficient angiogenesis and thus promoting vascular remodeling (Machado R D 2001, Teichert-Kuliszewska K 2006, de Jesus Perez V A 2009389). While the exact molecular mechanisms remain unclear, and technologies are advancing that now allow for more direct investigation of BMPR2 deficiency directly in patients with predisposing mutations. Gu and colleagues (Gu M, 2017) recently studied endothelial cells differentiated from human inducible pluripotent stem cells (iPSC-ECs) derived from 3 wild-type controls and 8 BMPR2-mutation positive carriers across 3 hereditary PAH families Transcriptomic sequencing across these cells previously yielded insights about certain BIRC3-specific signaling pathways, but a comprehensive validation of all BMPR2-relevant factors differentially modulated with these mutations was not performed.

In the present study, the publicly available RNA sequencing dataset was re-analyzed to determine that the transcript for the Signal Peptide CUB-EGF-Domain Containing Protein 1 (SCUBE1) was differentially expressed in iPSC-ECs carrying BMPR2 mutations. The protein structure of SCUBE1 carries both BMP1 and EGF domains and has been proposed as a direct BMP co-receptor (Tu C F, 2008). Increased circulating plasma SCUBE1 levels have been related to thromboembolic events, such as ischemic or hemorrhagic stroke, acute coronary syndrome, pulmonary embolism and deep vein thrombosis (Dai D F, 2008; Wu M Y, 2014; Turkmen S, 2014), but the role of SCUBE1 in PAH has not been described. Guided by such sequencing, SCUBE1 was identified as a secreted factor downregulated by multiple triggers of PAH and integral in BMPR2-specific endothelial pathophenotypes. Decreased SCUBE1 was found to be specific in PAH patients and correlated with the clinical features of pulmonary remodeling in both PAH and PVH patients with high PVR.

### Example 2. RNA Sequencing Data from BMPR2-Mutant iPSC-ECs Identify SCUBE1 as a Factor Integrally Linked to PAH Pathogenesis

Publicly available RNA sequencing (RNA Seq) data of human inducible pluripotent stem cells (iPSC-ECs) derived from 3 wild-type controls and 8 BMPR2-mutation positive carriers across 3 hereditary PAH families (Gu M, 2017) were analyzed via Salmon (Patro R, 2017) and DESeq2 with the intent to define differentially expressed genes in BMPR2-mutant cells after adjustment for false discovery rate. As shown in Table 1, 17 transcripts were identified as being significantly differentially expressed between BMPR2 mutant and wildtype controls (|log₂(fold change)|>1.5 and adjusted P-value <0.05). Of these transcripts, a systematic literature search revealed functional relevance to BMP signaling for 2 genes (SCUBE1 and MX1) (Yuan H, 2016; Yang R B, 2002) and relevance to the related transforming growth factor (TGF) superfamily pathway for 1 gene, CDH6 (Sancisi V, 2013). In contrast to the more indirect downstream signaling connections reported for MX1 and CDH6, SCUBE1, which encodes Signal Peptide CUB-EGF-Domain Containing Protein 1, carries a unique molecular structure including both BMP1 and EGF domains and has been proposed as a direct BMP co-receptor (Tu C F, 2008). Such direct links to BMPR2 made SCUBE1 a promising candidate for further analysis in endothelial function and PAH pathogenesis.

TABLE 1
Genes differentially expressed in RNA sequencing
data from iPSC-ECs derived from affected BMPR2
mutant patients and wild-type controls.

| Gene | Adj. P-Value | Functional Relevance |
|---|---|---|
| FAM65B | 0.03 | RhoA related migration |
| CDH6 | 0.001 | TGF signaling (Sancisi V, 2013) |
| ST8SIA6 | 0.04 | PI3K/Akt signaling |
| SCUBE1 | 0.001 | BMP/TGF signaling (Tsao KC, 2013) |
| NRG3 | 0.006 | EGF-like signaling |
| IFI44L | <0.001 | Interferon Response |
| MX1 | 0.006 | BMP signaling (Yuan H, 2016) |
| | | Interferon Response |
| BST2 | 0.02 | Interferon Response |
| H19 | 0.02 | Non-coding RNA |
| C1QTNF3 | <0.001 | PKC signaling |
| RARB | 0.003 | DNA demethylation |
| IFI6 | 0.02 | Interferon Response |
| IFIT1 | 0.02 | Interferon Response |
| CRHBP | 0.02 | Chromosome segregation |
| CALN1 | 0.004 | Calcium-binding protein |
| TMEM200C | 0.03 | Unknown function |
| DBF4P1 | 0.004 | Pseudogene |

### Example 3. Genetic and Acquired PAH Triggers Modulate SCUBE1 Expression in Cultured Pulmonary Arterial Endothelial Cells (PAECs)

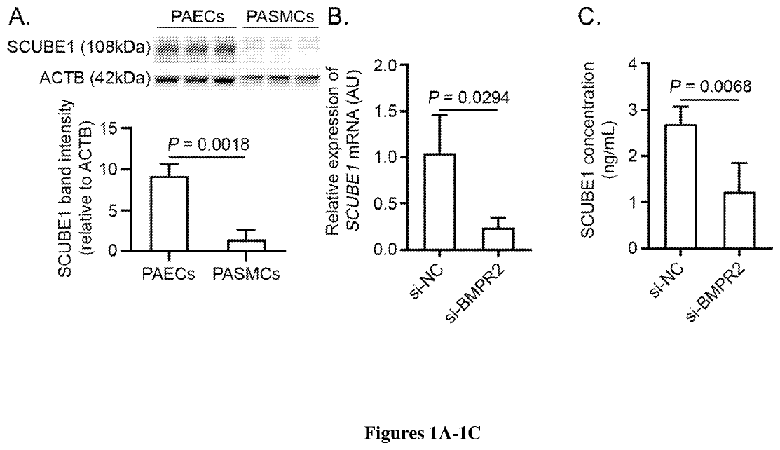

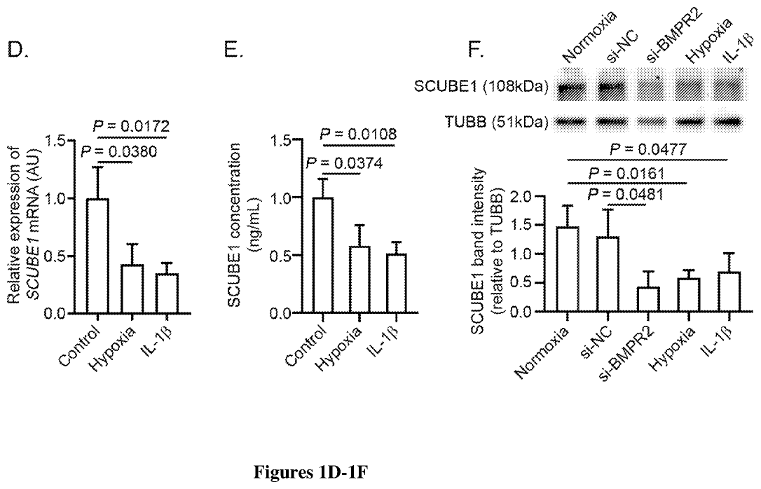

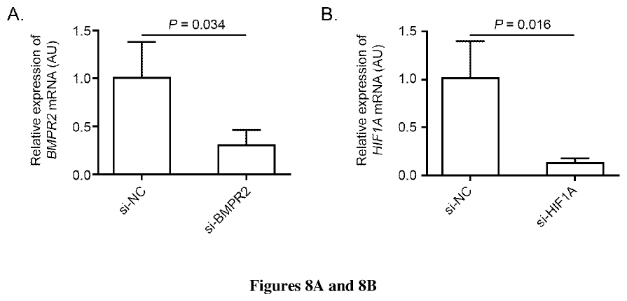

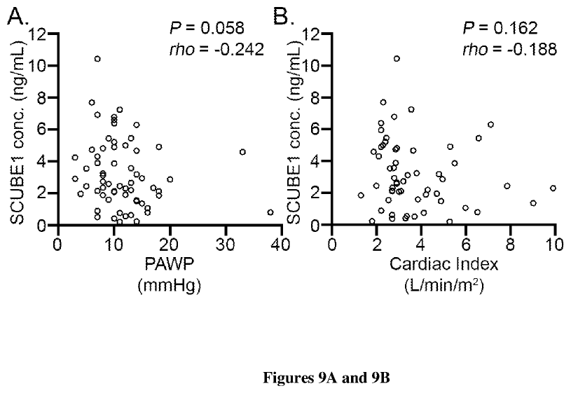

As shown in FIG. 1A, immunoblotting demonstrated substantial expression of SCUBE1 in PAECs but not in PA smooth muscle cells (PASMCs), suggesting endothelial-specific enrichment in the pulmonary vasculature. In cultured PAECs, the effects on SCUBE1 expression of various genetic and acquired triggers of PAH were examined Chronic exposure to inhibitory RNA (siRNA) knockdown of BMPR2 in PAECs significantly downregulated SCUBE1 at transcript, secreted, and intracellular protein levels (FIGS. 1B to 1C and FIG. 1F; FIG. 8A showing the efficacy of siRNA knockdown on BMPR2 transcript). The downregulation in SCUBE1 across transcript, secreted protein, and intracellular protein levels was also observed in PAECs treated with hypoxia or the inflammatory cytokine interleukin-1β (IL-1β), two well-known acquired triggers for PAH (FIGS. 1D to 1E and FIG. 1F).

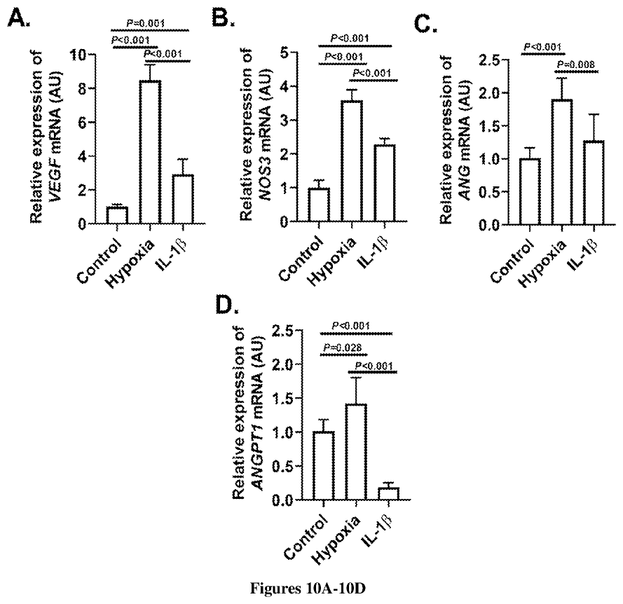

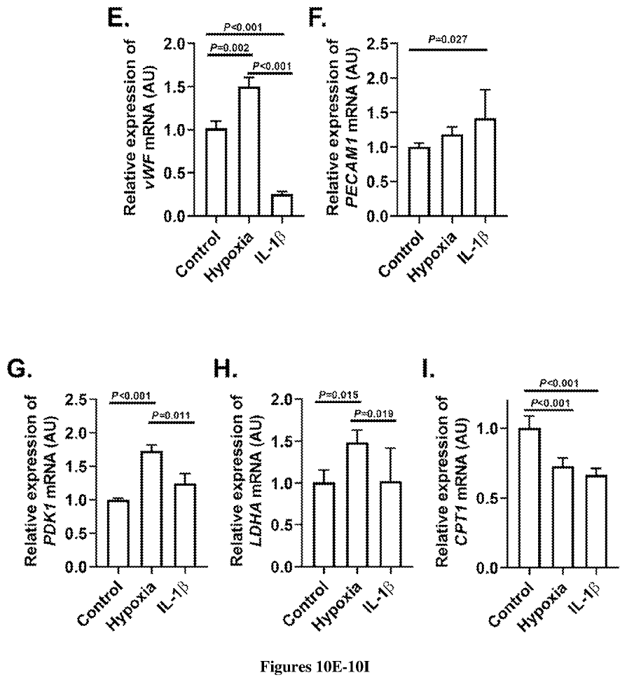

To rule out that downregulation of SCUBE1 by specific PAH triggers is secondary to a general suppression of PAEC activity and viability, the levels of known regulators of endothelial function in PAECs exposed to hypoxia and IL-1β were quantified. The expression of angiogenesis-, proliferation- and apoptosis-related genes ANG, VEGF, NOS3, and ANGPT1, adhesion molecule genes VECAM1 and VWF, and endothelial metabolism-associated genes PDK1, LDHA, CPT1, were profiled. As shown in FIG. 10, most endothelial genes examined were upregulated by hypoxia (VEGF, NOS3, ANG, ANGPT1, vWF, PDK and LDHA), with a downregulation of CPT1 only and no significant change to PECAM1. When PAECs were treated with IL-1β, the expression of VEGF, NOS3 and PECAM1 was increased, whereas the expression of other genes was decreased (ANGPT1, vWF and CPT1) or remained unchanged (ANG, PDK1 and LDHA). In total, these findings demonstrated an expected and specific set of reprogramming events in viable endothelium in response to hypoxia and inflammatory stress. In this context, these results suggested that specific downregulation of SCUBE1 may coordinate with other stress-responsive signaling pathways to control endothelial function in the setting of PAH.

### Example 4. Hypoxia Induced Factor 1α (HIF-1α) Mediates the Downregulation of SCUBE1 by Hypoxia and IL-1β but not BMPR2 Knockdown

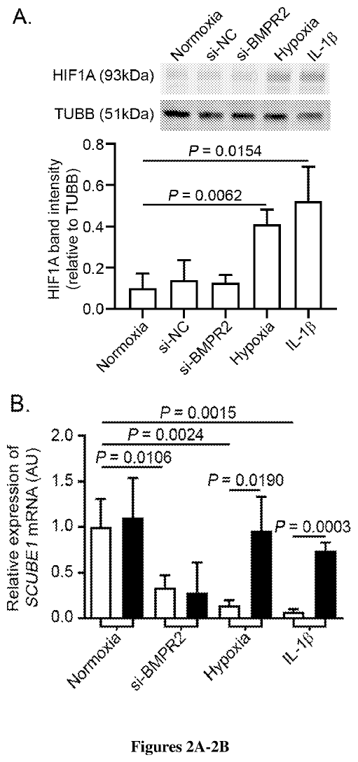

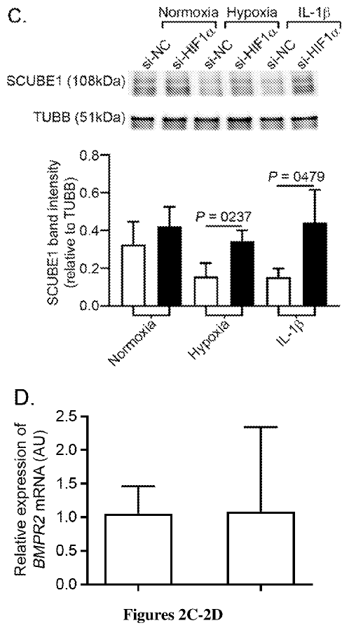

Hypoxia inducible factor-1 alpha (HIF-1α) is a master regulatory factor that controls hypoxic reprogramming in endothelial cells. In addition to true hypoxia, inflammatory cytokines such as IL-1β also induce HIF-1α accumulation (Jung Y J, 2003). To determine the involvement of HIF-1α in SCUBE1 downregulation by these acquired PAH triggers, HIF-1α siRNA knockdown was performed. The efficacy of siRNA knockdown of the HIF-1α gene in PAECs was confirmed by RT-qPCR (FIG. 8B). As shown in FIG. 2A, intracellular HIF-1α levels were increased in PAECs exposed to hypoxia or IL-1β but remained unchanged with BMPR2 siRNA knockdown. Correspondingly, while hypoxia and IL-1β consistently decreased SCUBE1 transcript levels in PAECs treated with nonspecific scrambled control RNA, HIF-1α knockdown nearly completely reversed SCUBE1 downregulation induced by hypoxia and IL-1β (FIGS. 2B and 2C), which appeared to be independent of BMPR2 expression as evidenced by the unchanged BMPR2 expression profile with HIF-1α knockdown (FIG. 2D). Conversely, consistent with the lack of dependence of HIF-1α expression on BMPR2, HIF-1α knockdown did not alter the extent of SCUBE1 downregulation driven by BMPR2 deficiency (FIG. 2C).

### Example 5. SCUBE1 Regulates Angiogenic Potential, Proliferation, and Apoptosis in Cultured PAECs

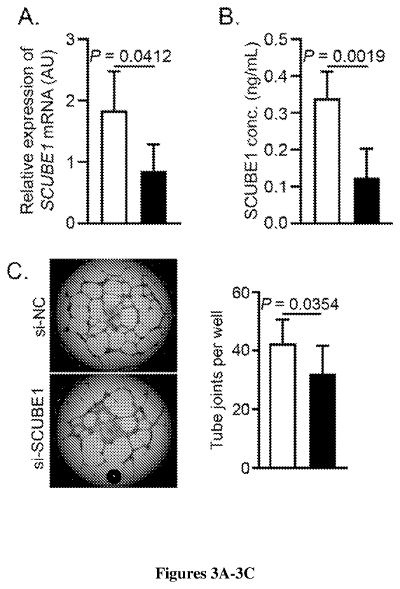

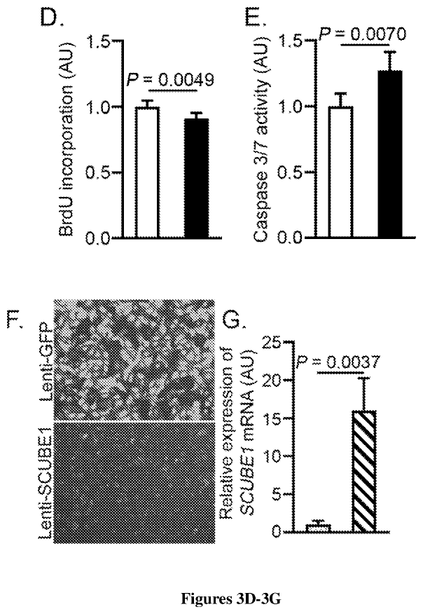

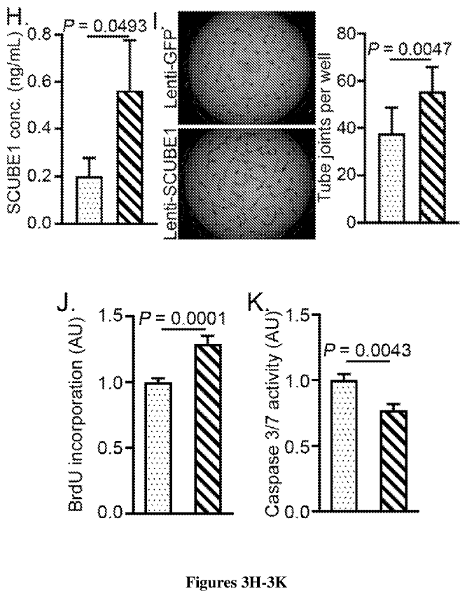

To determine the function of SCUBE1 in the control of PAEC activity, we conducted loss- and gain-of-function analyses with manipulation of SCUBE1 expression in PAECs by siRNA knockdown (FIGS. 3A and 3B) and forced SCUBE1 expression with lentiviral transduction of a SCUBE1 transgene, respectively (FIGS. 3F to 3H). SCUBE1 knockdown in PAECs significantly inhibited tube formation of PAECs in Matrigel (FIG. 3C), inhibited PAEC proliferation as determined by BrdU incorporation (FIG. 3D), and increased apoptosis as indicated by caspase 3/7 activity (FIG. 3E). Conversely, forced SCUBE1 overexpression in PAECs enhanced tube formation (FIG. 3I), increased proliferation (FIG. 3J), and decreased apoptosis (FIG. 3K). Taken together, these results demonstrate that SCUBE1 is both necessary and sufficient to invoke a protective function against PAH in the pulmonary endothelium, controlling a pro-angiogenic effect and augmenting survival and proliferative capacity.

### Example 6. SCUBE1 Controls the Activation of BMPR2 Associated SMAD Signaling Mediators

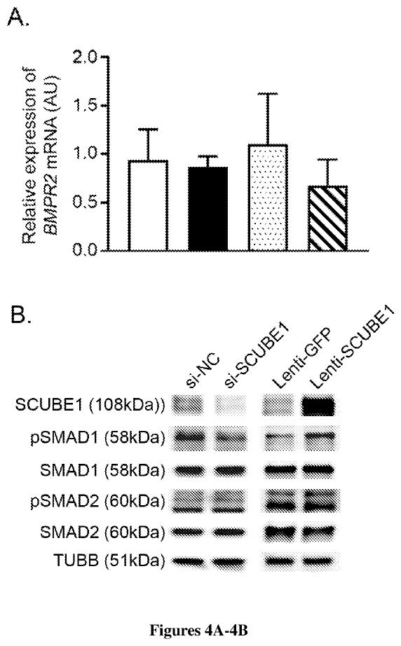

Prior reports have suggested SCUBE1 may act as a binding partner and co-activator of BMPR2 receptor, via the BMP domain located at the N terminus of the protein (Tu C F, 2008). To clarify this functionality of SCUBE1 in PAECs, BMPR2- and TGF-β-specific SMAD signaling mediators were quantified under SCUBE1 knockdown or forced expression. As shown in FIG. 4A, although SCUBE1 knockdown or overexpression significantly altered the intracellular SCUBE1 protein in PAECs, it did not change BMPR2 transcript level. In contrast, such knockdown and forced expression of SCUBE1 significantly reduced and increased levels, respectively, of activated and phosphorylated Smad1/5/9 relevant to BMPR2 activation, respectively (FIGS. 4B and 4C). The manipulation of SCUBE1, either via siRNA knockdown or forced expression, had no influence on activated and phosphorylated SMAD2/3 relevant to TGF-β-relevant signaling (FIGS. 4B and 4D). These results support the notion that SCUBE1 functions primarily through BMPR2-relevant SMAD signaling in PAECs.

### Example 7. SCUBE1 Levels are Decreased in Plasma and Lung Tissue in PAH Rodent Models

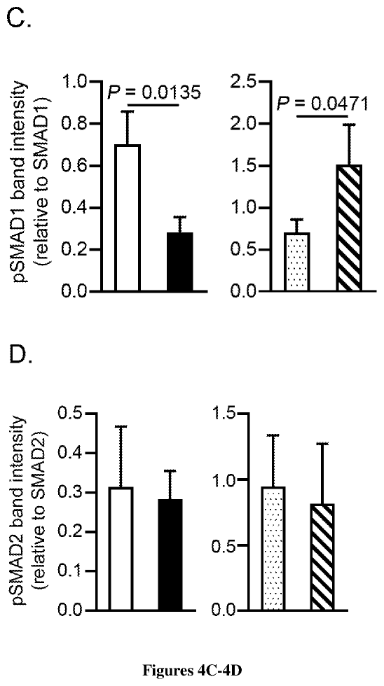

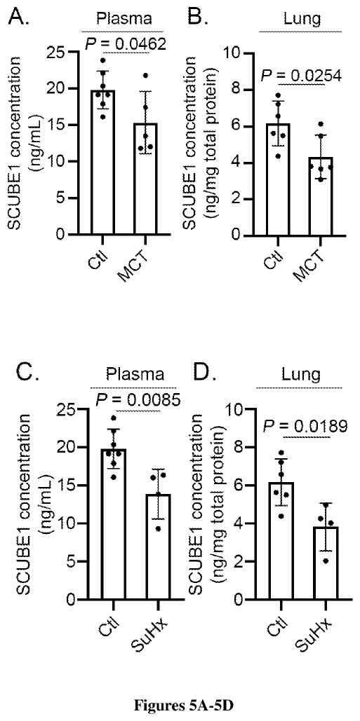

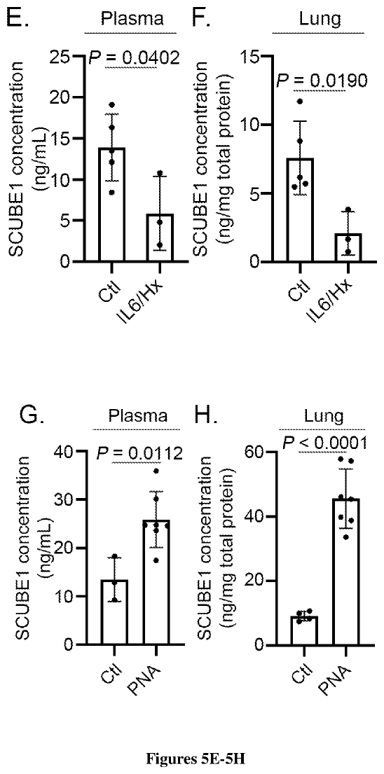

To determine the relevance of SCUBE1 regulation to PAH, secreted SCUBE1 levels in plasma and lung tissue homogenates were quantified by ELISA in three well-established PAH animal models: monocrotaline (MCT)-exposed or SU-5416-chronic hypoxia-exposed PAH rats, and pulmonary-specific interleukin-6 (IL-6) transgenic mice exposed to chronic hypoxia (Bertero T, 2014). As shown in FIGS. 5A-5F, in these three PAH rodent models, SCUBE1 levels were down-regulated significantly in both diseased plasma and lung tissues as compared with those in control animals.

To define the specificity of the SCUBE1 decrease compared with other rodent models of cardiopulmonary disease, plasma and tissue SCUBE1 levels were measured in mice with acute bacterial pneumonia induced by K. pneumoniae inoculation and acute myocardial infarction (AMI) induced by left coronary artery ligation. As shown in FIGS. 5G-5J, both plasma and organ tissue (lung or heart) SCUBE1 levels were significantly increased in the rodents with acute pneumonia or acute myocardial ischemia.

### Example 8. SCUBE1 Levels are Decreased in Plasma and Lung Tissue from PAH Patients

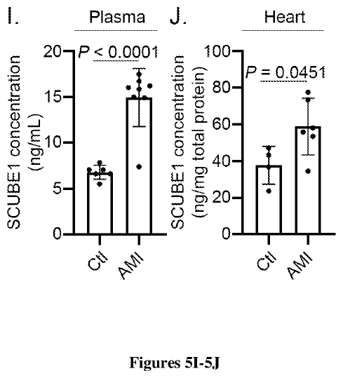

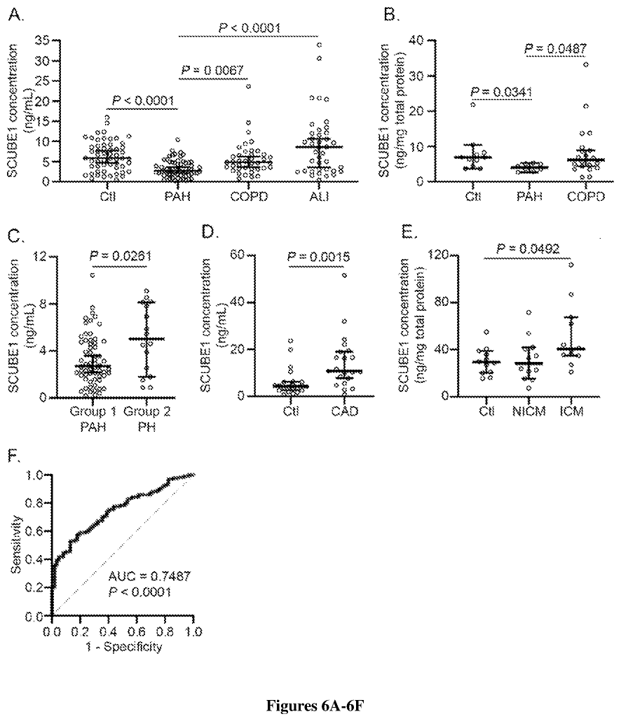

Also, because SCUBE1 is a secreted factor inherently relevant to endothelial pathophenotypes in PAH and based on the above rodent studies, differential plasma levels in humans can be utilized to distinguish PAH from other cardiopulmonary diseases. Plasma specimens were collected from 62 WSPH Group 1 PAH patients and 16 WSPH Group 2 PH patients at two separate U.S. PH referral centers, confirmed clinically and hemodynamically by invasive RHC. As comparisons, 56 non-PH individuals, 39 patients with chronic obstructive pulmonary disease (COPD), and 39 patients with acute lung injury (ALI) or clinical ARDS were included for plasma SCUBE1 measurement. Tables 2, 3, and 4 describe the demographics and available hemodynamic profiles of these study patients at the time of blood draw. As shown in FIG. 6A and quantified by ELISA, plasma SCUBE1 levels from PAH patients (median 2.70, Q1-Q3 range 1.80-4.76 ng/mL) were significantly lower than non-PAH controls (median 5.84, Q1-Q3 range 3.51-9.10 ng/mL, P<0.001), COPD patients (median 4.81, Q1-Q3 range 3.23-6.78 ng/mL, P=0.007), and ALI patients (median 8.61, Q1-Q3 range 2.65-12.17 ng/mL, P<0.001). Additionally, we collected lung tissues from rapid autopsy or transplant lungs derived from 8 PAH patients, 11 non-PAH controls, and 20 COPD patients. Demographics of tissue donors are listed in Table 5. As shown in FIG. 6B, correlating with decreased levels in PAH plasma, SCUBE1 levels in lung tissue from PAH patients (median 4.00, Q1-Q3 range 2.98-5.08 ng/mg tissue) were significantly lower than non-PAH controls (median 6.93, Q1-Q3 range 4.38-8.28 ng/mg tissue, P=0.034) and COPD patients (median 6.15, Q1-Q3 range 4.05-9.54 ng/mg tissue, P=0.049).

TABLE 2
PH, COPD and ALI patient demographics for plasma samples.

| | Non-diseased | Group 1 | Group 2 | | |
|---|---|---|---|---|---|
| Cohort | Controls | PAH | PH | COPD | ALI |
| n | 56 | 62 | 16 | 39 | 39 |
| Age (Mean ± SD, years) | 47.4 ± 17.6 | 58.4 ± 13.6 | 64.9 ± 11.1 | 63.5 ± 9.8 | 51.9 ± 15.9 |
| Gender (n and % female) | 37(66.1%) | 48(77.4%) | 9(56.3%) | 14(35.9%) | 18(46.2%) |
| Race (n and % white) | 38(67.9%) | 56(90.3%) | 14(87.5%) | 39(100%) | 38(97.4%) |

PH: pulmonary hypertension;
PAH: pulmonary arterial hypertension;
COPD: chronic obstructive pulmonary disease;
ALI: acute lung injury

TABLE 3
Patient hemodynamic parameters by PH classification.
Hemodynamic parameters are shown as median with 25th
and 75th (Q1-Q3) interquartile range. P-values were
calculated by Mann-Whitney nonparametric test.

| PH Group | Group 1 PAH | | Group 2 PH | | P-Value |
|---|---|---|---|---|---|
| n | 62 | | 16 | | |
| mean PAP (mmHg) | 42.0 | (32.8-53.0) | 42.5 | (37.5-52.3) | 0.521 |
| PCWP (mmHg) | 10.0 | (9.0-12.0) | 22.5 | (17.0-27.0) | <0.001 |
| CO (Fick, L/min) | 5.3 | (4.5-6.8) | 5.8 | (5.1-7.9) | 0.193 |
| PVR (WU) | 5.2 | (3.8-7.1) | 3.1 | (1.9-4.4) | <0.001 |

PH: pulmonary hypertension; PAH: pulmonary arterial hypertension; PAP: pulmonary artery pressure; PCWP: pulmonary capillary wedge pressure; CO: cardiac output; PVR: pulmonary vascular resistance; WU: Wood units.

TABLE 4
Demographics and hemodynamic parameters of
Group 1 PAH patients at time of blood draw.

| | | | | mean PAP | PVR |
|---|---|---|---|---|---|
| Etiology | Age | Gender | Race | (mmHg) | (WU) |
| Idiopathic | 39 | Female | White | 45 | 3.8 |
| Portal | 63 | Male | White | 56 | 9.1 |
| hypertension | | | | | |
| Idiopathic | 59 | Female | White | 36 | 4 |
| Scleroderma | 70 | Female | White | 44 | 9.4 |
| Congenital | 40 | Male | White | 31 | 5.2 |
| heart disease | | | | | |
| Scleroderma | 72 | Female | White | 27 | 5.2 |
| Scleroderma | 73 | Female | White | 41 | 6.4 |
| Scleroderma | 65 | Female | White | 32 | 3.8 |
| Idiopathic | 57 | Male | White | 53 | 9.1 |
| Idiopathic | 26 | Female | White | 34 | 5.4 |
| Scleroderma | 73 | Female | White | 32 | 3.8 |
| Scleroderma | 37 | Male | White | 37 | 3.4 |
| Dermatomyositis | 68 | Male | White | 35 | 5.3 |
| Idiopathic | 43 | Female | White | 52 | 11.2 |
| Idiopathic | 36 | Female | White | 36 | 7 |
| HIV | 48 | Male | White | 72 | 16 |
| Scleroderma | 71 | Female | White | 33 | 4.8 |
| Idiopathic | 53 | Male | White | 27 | 4.7 |
| Idiopathic | 67 | Male | White | 54 | 6.6 |
| Scleroderma | 64 | Female | White | 46 | 6.6 |
| Scleroderma | 72 | Female | White | 26 | 2.5* |
| Idiopathic | 75 | Female | White | 42 | 3.1 |
| Scleroderma | 43 | Female | Black | 27 | 2.7* |
| Scleroderma | 75 | Female | White | 51 | 6.4 |
| Scleroderma | 73 | Female | White | 57 | 8.5 |
| Idiopathic | 66 | Female | White | 54 | 6.9 |
| Scleroderma | 76 | Female | Black | 39 | 3.2 |
| Idiopathic | 70 | Female | White | 29 | 4.3 |
| Idiopathic | 76 | Female | White | 42 | 11 |
| Scleroderma | 86 | Female | White | 42 | 8.4 |
| Congenital | 36 | Female | White | 58 | 7.5 |
| heart disease | | | | | |
| Scleroderma | 57 | Female | White | 27 | 2.5* |
| Systemic | 73 | Female | White | 38 | 5 |
| sclerosis | | | | | |
| Idiopathic | 53 | Female | White | 56 | 13 |
| Scleroderma | 58 | Female | White | 53 | 6.8 |
| Scleroderma | 65 | Female | White | 47 | 6.9 |
| Scleroderma | 47 | Female | White | 32 | 2.2* |
| Idiopathic | 27 | Female | White | 60 | 4.9 |
| Congenital | 39 | Female | White | 40 | 4.2 |
| heart disease | | | | | |
| Connective | 83 | Female | White | 41 | 5 |
| tissue disease | | | | | |
| Idiopathic | 58 | Female | White | 53 | 5.5 |
| Idiopathic | 47 | Male | White | 52 | 11.7 |
| Idiopathic | 53 | Female | White | 54 | 5.1 |
| Connective | 57 | Female | White | 65 | 5.7 |
| tissue disease | | | | | |
| Idiopathic | 73 | Female | White | 28 | 3.6 |
| Idiopathic | 41 | Male | White | 51 | 2.9* |
| Connective | 61 | Female | White | 25 | 4.1 |
| tissue disease | | | | | |
| Idiopathic | 55 | Female | Black | 44 | 3.4 |
| Connective | 58 | Female | White | 53 | 3.6 |
| tissue disease | | | | | |
| Idiopathic | 58 | Male | White | 61 | 4.2 |
| Connective | 58 | Male | White | 43 | 3 |
| tissue disease | | | | | |
| Idiopathic | 55 | Female | White | 50 | 6.5 |
| Scleroderma | 62 | Male | White | 33 | 4.5 |
| Scleroderma | 61 | Male | White | 25 | 3.1 |
| Scleroderma | 56 | Female | White | 38 | 5 |
| Scleroderma | 51 | Female | Black | 39 | 9.2 |
| Idiopathic | 59 | Female | White | 56 | 13.1 |
| Scleroderma | 50 | Female | White | 23* | 1.5* |
| Idiopathic | 71 | Female | White | 45 | 7.7 |
| Congenital | 43 | Female | White | 53 | 5.6 |
| heart disease | | | | | |
| Connective | 55 | Female | White | 52 | 7.5 |
| tissue disease | | | | | |
| Idiopathic | 63 | Female | White | 20* | 3.8 |

*The diagnosis was made based on prior invasive hemodynamics measurement, which fulfilled the criteria for Group 1 PAH.
PAP: pulmonary artery pressure; PVR: pulmonary vascular resistance; WU: Wood units.

TABLE 5
Patient demographics for lung tissue donors.

| | Non-diseased | | |
|---|---|---|---|
| Cohort | controls | PAH | COPD |
| n | 11 | 8 | 20 |
| Age (Mean ± SD, years) | 46.6 ± 12.8 | 52.8 ± 16.7 | 62.1 ± 9.6 |
| Gender (n and % female) | 5(45.5%) | 7(87.5%) | 8(40.0%) |
| Race (n and % white) | 8(72.7%) | 7(87.5%) | 17(85.0%) |

PAH: pulmonary arterial hypertension; COPD: chronic obstructive pulmonary disease.

In contrast to these findings in PAH patients, plasma SCUBE1 levels in WSPH Group 2 PH patients (median 5.02, Q1-Q3 1.98-8.06 ng/mL) were, on average, significantly higher than PAH patients (P=0.026, FIG. 6C). Patients' demographics and hemodynamics were listed in Tables 2 and 3. Beyond lung disease, further clarification of the specificity of decreased SCUBE1 expression in plasma was sought, as SCUBE1 is known to change in the setting of ischemic heart disease (Dai D F et al., 2008) SCUBE1 levels were quantified in serum samples from 21 patients with coronary artery disease (CAD) confirmed by coronary angiography vs. 22 patients without obstructive CAD. In parallel, SCUBE1 was also measured from left ventricular myocardial samples isolated from 12 non-ischemic cardiomyopathy (NICM) patients and 12 ischemic cardiomyopathy (ICM) patients (Tables 6 and 7 for patient demographics). As shown in FIGS. 6D-6E, SCUBE1 serum levels were significantly higher in CAD patients and in heart tissues from ICM patients, when comparing to those from non-CAD controls.

TABLE 6
CAD patient demographics for serum samples.

| | Non-CAD | |
|---|---|---|
| Cohort | controls | CAD |
| n | 22 | 21 |
| Age (Mean ± SD, years) | 60.8 ± 9.9 | 61.3 ± 11.0 |
| Gender (n and % female) | 8 (26.7%) | 6 (28.6%) |
| Race (n and % white) | 19 (86.4%) | 20 (95.2%) |

CAD: coronary artery disease.

TABLE 7
Patient demographics for heart tissue donors.

| | Non-diseased | | |
|---|---|---|---|
| Cohort | controls | ICM | NICM |
| n | 12 | 12 | 12 |
| Age (Mean ± SD) | 52.6 ± 10.6 | 61.4 ± 8.3 | 49.3 ± 10.0 |
| Gender (n and % female) | 2(16.7%) | 0(0.0%) | 6(50.0%) |
| Race (n and % white) | 9(75.0%) | 11(91.7%) | 8(66.7%) |

ICM: ischemic cardiomyopathy; NICM: non-ischemic cardiomyopathy.

To evaluate the diagnostic value of plasma SCUBE1 measurement in PAH, we performed a Receiver Operating Characteristic (ROC) analysis between PAH and a combined non-PAH cohort composed of control, COPD, and ALI subjects (FIG. 6F), resulting in an AUC of 0.75 (P<0.001). An optimal plasma SCUBE1 cut point of 5.46 ng/mL was defined to distinguish PAH from the non-PAH cohorts with a high specificity of 0.87 and a sensitivity of 0.53 (Table 8 for summary of statistics). Furthermore, the diagnostic odds ratio (OR), a single indicator of diagnostic performance, was calculated as previously described (27). The diagnostic OR for a plasma SCUBE1 cut point of 5.46 ng/mL was 7.6 (95% confidence interval (CI) 3.4-16.9, P<0.001) to diagnose PAH against non-PAH controls. We also performed a ROC analysis between Group 1 PAH and Group 2 PH cohorts, resulting in an AUC 0.68 (P=0.027). To discriminate between these PH subtypes, an optimal plasma SCUBE1 cut point of 5.01 ng/mL was defined, again with a high specificity of 0.82 and a sensitivity of 0.50 (Table 9 for summary of statistics). The diagnostic OR for this plasma SCUBE1 cut point was 4.6 (95% CI 1.5-14.7, P=0.011) to diagnose Group 1 PAH against Group 2 PH.

TABLE 8
Summary of statistics for ROC analysis between PAH and a combined
non-PAH cohort composed of control, COPD, and ALI subjects.

| | |
|---|---|
| AUC area (C-statistics) | 0.75 |
| Standard error | 0.035 |
| 95% confidence interval | 0.681-0.817 |
| P value of AUC area | <0.001 |
| Sensitivity of optimal | 0.53 |
| SCUBE1 cut point 5.46 ng/mL | |
| 95% confidence interval | 0.45-0.61 |
| Sensitivity of optimal | 0.87 |
| SCUBE1 cut point 5.46 ng/mL | |
| 95% confidence interval | 0.77-0.93 |
| Likelihood ratio | 4.11 |

TABLE 9
Summary of statistics for ROC analysis between
WSPH Group 1 PAH and Group 2 PH cohorts.

| | |
|---|---|
| AUC area (C-statistics) | 0.68 |
| Standard error | 0.085 |
| 95% confidence interval | 0.515-0.846 |
| P value of AUC area | 0.027 |
| Sensitivity of optimal | 0.50 |
| SCUBE1 cut point 5.01 ng/mL | |
| 95% confidence interval | 0.28-0.72 |
| Sensitivity of optimal | 0.82 |
| SCUBE1 cut point 5.01 ng/mL | |
| 95% confidence interval | 0.71-0.90 |
| Likelihood ratio | 2.82 |

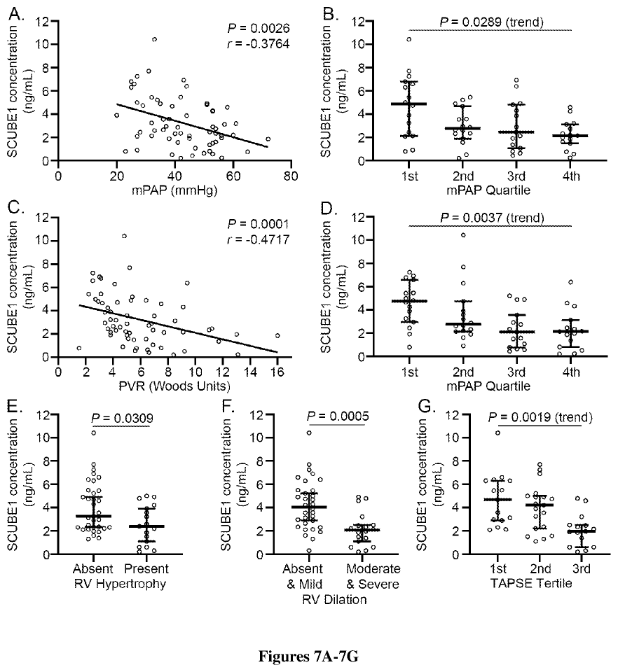

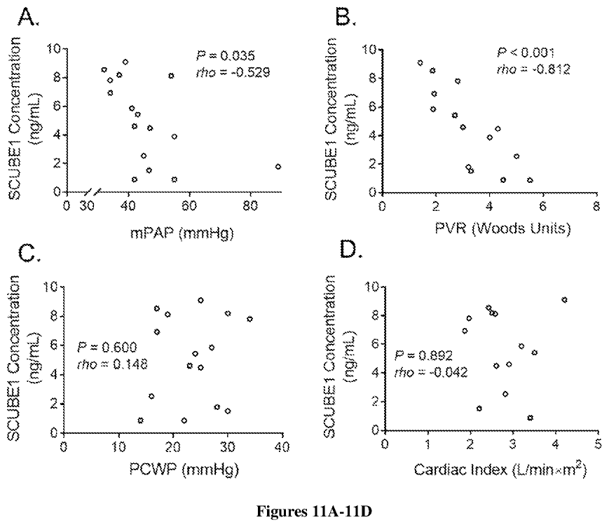

If SCUBE1 expression is correlated with hemodynamic or echocardiographic parameters linked to severity of PH was determined. In PAH patients, plasma SCUBE1 levels were found to be progressively reduced with increasing levels of either mean pulmonary artery pressure (mPAP) or pulmonary vascular resistance (PVR), with statistical significance in both regression analysis (FIGS. 7A and 7C) and the trend of decrease analysis when binned mPAP and PVR into quartiles (FIGS. 7B and 7D). The significant negative correlation between plasma SCUBE1 levels and hemodynamic parameters reflecting the severity of pulmonary vascular remodeling was also observed in WSPH Group 2 PVH patients (FIGS. 11A for mPAP and 11B for PVR). No correlation was found between pulmonary capillary wedge pressure (PCWP) or cardiac output with plasma SCUBE1 levels in either WSPH Group 1 PAH or Group 2 PVH patients (FIG. 9 and FIG. 11C-11D). When examining the UPMC cohort of patients, plasma SCUBE1 levels were also significantly lower in PAH patients with echocardiographic indices of severe PAH, including right ventricle (RV) hypertrophy, moderate to severe RV dilation, or decreased tricuspid annular plane systolic excursion (TAPSE), a quantitative echocardiographic measurement reflecting RV dysfunction (FIGS. 7E-7G).

### Example 9. The Use of SCUBE1 for Treating and Diagnosing Pulmonary Arterial Hypertension

The complete molecular mechanisms that predispose persons carrying BMPR2 heterozygous mutations to PAH are not entirely defined, but iPSC technologies now allow for more direct investigation of BMPR2 deficiency in patients with predisposing mutations. By analyzing a publicly available RNA-sequencing dataset generated from iPSC-ECs of BMPR2-mutant carriers and PAH patients, SCUBE1 was identified as a BMPR2-relevant secreted factor regulated by multiple triggers of PAH and which modulates crucial endothelial pathophenotypes in PAH. The mechanistic relationship and interaction of PAH triggering factors and signaling molecules with SCUBE1 in PAECs are summarized in FIG. 13. Correspondingly, in multiple PAH rat models and PAH patients, SCUBE1 levels were decreased and negatively correlated with the severity and progression of disease, suggesting the potential of developing SCUBE1 as a diagnostic and prognostic marker for this historically neglected disease.

In the past two decades, genomic and mechanistic studies have defined BMPR2 biology as a genetic (Machado R D, 2009) and molecular (Johnson D W, 1996; McAllister K A, 1994; Shintani M, 2009) lynchpin of PAH pathogenesis, but substantial knowledge gaps still exist. Pathogenic BMPR2 mutations are genetically diverse (Austin E D, 2014) but produce a common haploinsufficiency which has been generally accepted as a driver of endothelial dysfunction, vascular remodeling, and vasoconstriction ultimately leading to clinical PAH (Machado R D, 2001). While inherited in an autosomal dominant fashion, the incomplete penetrance of roughly 20% in families harboring pathogenic BMPR2 mutations reveals the current limited understanding of the complex genetic and environmental interactions behind its clinical manifestation (Larkin E K, 2012). Part of the knowledge deficiency regarding BMPR2 biology can be attributed to the difficulty of recapitulating its haploinsufficiency using traditional knockdown experiments. Gu and colleagues (Gu M, 2017) sought to bypass this obstacle by harnessing induced pluripotent stem cell (iPSC) technology in which RNA-Sequencing was performed on transcripts isolated from iPSC endothelial cells (iPSC-ECs) derived from PAH patients with BMPR2 mutations, unaffected carriers of BMPR2 mutations, and healthy controls. Rapid advances in analytical methods over the past several years, notably correction for G-C content (Love M I, 2016), enabled to re-interrogate the data with improved sensitivity for detecting differentially expressed genes at a comparable false discovery rate (Patro R, 2017).

Besides SCUBE1, the RNA-Seq analysis disclosed herein also identified 16 other genes that were differentially expressed between iPSC-ECs from BMPR2 mutants versus controls. Most differentially expressed transcripts were messenger RNAs, with one notable long non-coding RNA, H19, that has been recently connected to PAH pathogenesis (Wang R, 2018; Su H, 2018). Furthermore, other genes have been linked to the interferon response, including BST2 (Blasius A L, 2006), IFIT1, IFI6, IFI44L, and MX1 (Yuan H, 2016). Clinical reports of PAH onset after interferon therapy in diseases such as hepatitis C and multiple sclerosis have indicated that interferon may play a role in PAH pathogenesis, although pre-clinical studies have yielded conflicting results as to whether type I interferons play a therapeutic (Bauer E M, 2014) or pathogenic (George P M, 2014) role in PAH. While it is possible that distinct interferon-related profiles can represent artifacts of the iPSC-EC differentiation process (Eggenberger J, 2019; Khan K A, 2015), MX1 has been linked to BMP signaling (Yuan H, 2016), providing internal validation of our approach and indicating that the interaction of interferon-associated signaling pathways with BMP is deserving of additional interrogation. Alternatively, the distinct interferon-related profiles can represent artifacts of the iPSC-EC differentiation process (Eggenberger J, 2019; Khan K A, 2015).

The functional and structural interconnections of SCUBE1 and BMPR2 offer substantial insights into the molecular pathobiology of both of these molecules. SCUBE1 is a secreted and cell-surface protein which consists structurally of an NH2-terminal signal peptide sequence, an EGF-like repeat domain, a spacer region, cysteine rich motifs, and a COOH-terminal CUB (Complement protein C1r/C1s, Uegf, and BMP1) domain, where expression is restricted mainly to platelets and endothelium during adulthood (Tu C F, 2008; Yang R B, 2002; Grimmond S, 2000). SCUBE1 deficiency was shown to recapitulate phenotypes associated with BMPR2 deficiency, including decreased angiogenic potential, and increased apoptosis (Wang H, 2014; Sa S, 2017). Meanwhile, SCUBE1 overexpression displayed converse effects and reversed the phenotypes associated with multiple known PAH triggers in vitro. Interestingly, SCUBE1 was shown to act as co-activator or interactor to both TGFβ receptor and BMPR2 (Tsao K C, 2013); the functional status and balance of these two cell-signaling systems are critical in control of endothelial function in PAH (Rol N, 2018). Examination of BMPR2 and TGFβ receptor-specific SMADs indicates that SCUBE1 preferentially controls SMADs more relevant to BMPR2 rather than TGFβ (FIG. 4) Given the finding that BMPR2 knockdown can also downregulate SCUBE1 (FIG. 1), the data herein support a model whereby SCUBE1 and BMPR2 form a positive feedback loop whereby deficiency of either one of the two partners may transform the loop into a vicious cycle to further downregulate the overall BMPR2 functional status in endothelial cells. Conversely, the enhancement of either SCUBE1 or BMPR2 can result in augmented functional regulation due to positive feedback to each other. These data endorse a use of SCUBE1 as a therapeutic target to effectively reinstall or augment BMPR2 signaling-related endothelial function during initiation and/or development of PAH.

Beyond BMPR2 deficiency, hypoxia and inflammatory factor IL-1β, two commonly recognized acquired triggering factors for PAH, also downregulated SCUBE1 (FIG. 1) with substantial dependence on HIF-1α (FIG. 2). Notably, HIF-1α likely employs an indirect mechanism for downregulation, given the lack of any known HIF-1α binding consensus motif ([A/G]CGTG) (Kimura H, 2001) in the SCUBE1 promoter region (data not shown). Furthermore, at baseline, HIF-1α siRNA knockdown did not alter BMPR2 expression and exerted no reversal effect on the downregulation of SCUBE1 by BMPR2 siRNA. Thus, a HIF-1α-independent mechanism must also exist, relevant to BMPR2-dependent effects on SCUBE1.

The present findings of circulating SCUBE1 plasma levels in PAH patients that inversely correlate with disease severity emphasize the putative diagnostic and/or prognostic utility of this molecule. Owing to its role in platelet aggregation and thrombosis (Tu C F, 2008; Wu M Y, 2014; Tu C F, 2006), SCUBE1 has been proposed as a plasma biomarker for myocardial infarction, thrombotic stroke, and pulmonary embolism (Dai D F, 2008, Turkmen S, 2015). The present study extensively tested the change of SCUBE1 levels in multiple acute and chronic cardiopulmonary pathologies, including pneumonia, ALI or ARDS, acute MI, COPD, chronic stable CAD, ICM, and NICM. Importantly, in these other cardiopulmonary diseases, increased, rather than decreased, plasma SCUBE1 was associated with disease state, thus offering substantial specificity of the present findings to PAH. This was reflected by the ROC analysis as well as a high specificity of 0.87 and a significant diagnostic odds ratio of 7.6 at the optimal plasma SCUBE1 cut point of 5.46 ng/mL. These findings support the notion that decreased plasma SCUBE1 is effective in distinguishing the presence of PAH over benign contexts and other non-PAH cardiopulmonary conditions. The great need for a diagnostic and prognostic biomarker in PAH is highlighted by the fact that clinical symptoms often manifest late in the course of the disease when right heart failure is evident, thus delaying medical therapy (Brown L M, 2011). Meanwhile, survival times from PAH diagnosis have more than doubled since the advent of advanced therapies (D'Alonzo G E, 1991; Benza R L, 2012), indicating that earlier application of these regimens can yield additional improvements in mortality. To date, however, the majority of candidate biomarkers have focused on non-specific indicators of right ventricular failure, angiogenesis, or inflammation (Anwar A, 2016). Recently, the identification of BMP9 as a clinical biomarker for portopulmonary hypertension emphasized the need for mechanistic biomarkers reflective of the underlying, and potentially genetic, pathophysiology (Nikolic I, 2019). While prior work has shown that plasma levels of soluble endoglin, a co-receptor involved in BMP signaling, are elevated in PAH and predict functional class (Malhotra R, 2013), these measurements have not been shown to correlate with hemodynamic parameters. In contrast, the study shown herein found that plasma SCUBE1 levels are tightly and negatively correlated with mean pulmonary artery pressures and pulmonary vascular resistance as well as indices of RV dysfunction.

The work shown herein also indicates that the diagnostic value of SCUBE1 can be especially evident in differentiating WSPH Group 1 PAH from Group 2 PH. Often, Group 2 PH can clinically masquerade as Group 1 PAH. As such, inappropriate pulmonary vasodilator treatment may be considered for Group 2 PH patients when diagnostic criteria are blurred (Maron B A, 2019). The decreased SCUBE1 levels observed in Group 1 PAH, but not Group 2 PH, in this initial study begin to clarify the distinct pathogenetic features between these two clinical groups. In this context, the discriminatory performance of SCUBE1 levels was modest (AUC 0.68), driven by the known heterogeneity across Group 2 PH patients and particularly by the wide range of pulmonary vascular resistance. Finally, beyond these PH groups, additional studies examining the association of plasma SCUBE1 levels with WSPH Group 3 (PH related to lung disease and hypoxia) and Group 4 PH (chronic thromboembolic pulmonary hypertension, or CTEPH), is of great interest, given the potential for platelet-released SCUBE1 to contribute to the directional gradient of the proposed biomarker.

In addition to differentiating Group 1 vs Group 2 PH in general, the prognostic value of SCUBE1 may extend to differentiating subtypes of the more prevalent Group 2 PH associated with left heart disease. Although SCUBE1 levels were higher at baseline in Group 2 versus Group 1 PH, SCUBE1 levels in Group 2 PH maintained a significant negative correlation with mPAP and PVR. Group 2 PH is hemodynamically defined by pulmonary hypertension associated with elevated left atrial filling pressures, and it can be further subclassified into combined pre- and post-capillary PH (Cpc-PH) and isolated postcapillary PH (Ipc-PH) based on the presence or absence, respectively, of hemodynamically-significant pulmonary vascular remodeling (Simonneau G, 2019). Cpc-PH is associated with increased mortality (Miller W L 2013) and an unfavorable prognosis after cardiac transplantation, thereby limiting transplant candidacy (Costard-Jackle A 1992). However, the current methods for distinguishing Cpc-PH from Ipc-PH are the subject of ongoing debate and invariably require invasive right heart catheterization (Wright S P 2017, Gerges C 2013). Based on the association of SCUBE1 downregulation with endothelial dysfunction, plasma SCUBE1 levels decrease in Cpc-PH versus Ipc-PH.

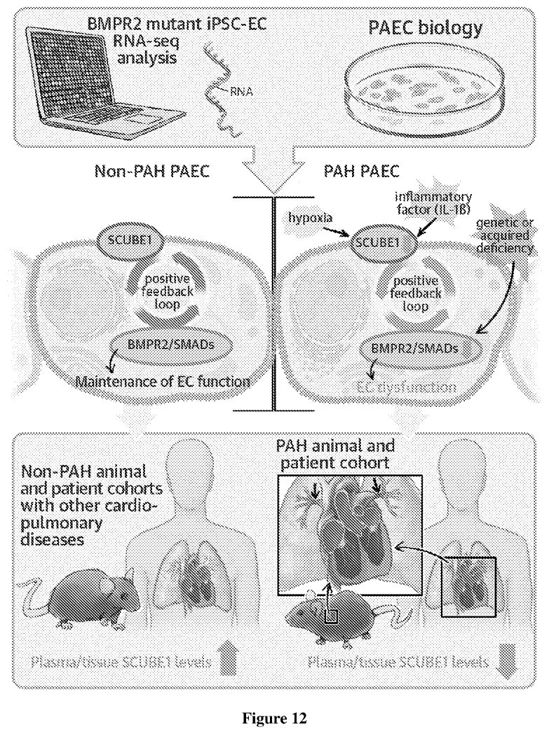

The present study shows that acquired triggers of pulmonary arterial hypertension (PAH), hypoxia and IL-1β upregulate HIF-1α and consequently downregulate SCUBE1 in pulmonary arterial endothelial cells (PAECs). Deficiency of BMPR2, either from genetic or acquired triggers, also downregulates SCUBE1. Decreased SCUBE1 modulates SMAD1/5/9 signaling downstream of BMPR2, thereby altering PAEC survival, proliferation, and angiogenic potential and leading to pulmonary vascular remodeling, PAH occurrence, and subsequent right heart failure (FIG. 12). Decreased plasma SCUBE1 correlates with indices of PAH, supporting its use as a clinical marker of disease.

### Example 10. Materials and Methods

RNA-Sequencing analysis. The RNA-Sequencing dataset from inducible pluripotent stem (iPS) cell-derived endothelial cells with and without BMPR2 mutations (Gu M, 2017) was available at GEO Series accession number GSE79613. Transcript abundances were quantified using Salmon (Patro R, 2017), and the tximport package (Soneson C, 2015) was used to assemble estimated count and offset matrices for the R package DESeq2 version 1.20.0 (Love M I, 2014) was used to identify differentially expressed genes were defined by adjusted P-value <0.05 and |log 2(fold change)|>1.5.

RT-qPCR and immunoblotting. RNA extraction, reverse transcription, and quantitative PCR (RT-qPCR) were performed as we previously described (Bertero T, 2014). Quantitative PCR was performed on an Applied Biosystems Quantstudio 6 Flex Fast Real Time PCR device. Fold-change of RNA species was calculated using the formula 2\hat{ }(−ΔΔCt), normalized to β-actin expression. SYBR qPCR primers for human SCUBE1 and β-actin were purchased from Bio-Rad. Taqman qPCR primers for human BMPR2, HIF-1α, ANG, VEGF, NOS3, ANGPT1, LDHA, CPT1, PDK1, PECAM-1, VWF, and β-actin were purchased from Thermo Fisher Scientific.

For immunoblotting, cellular proteins were isolated using RIPA lysis buffer and separated by SDS-PAGE and transferred to PVDF membranes (Bio-Rad). Membranes were blocked in 5% non-fat milk or bovine serum albumin (BSA) in TBS buffer containing 0.1% Tween (TBST) and incubated in the presence of the primary at 4° C. overnight and then secondary antibodies for 1 hour at room temperature. After washing in TBST buffer, immunoreactive bands were visualized with the ECL system (Amersham Biosciences). The density of the bands was quantified by densitometric analysis using the NIH ImageJ software (rsb.info.nih.gov/ij/).

Primary antibodies for SCUBE1 (ab105358, 1:500) and HIF-1α (1:500) were obtained from Abcam; phospho-SMAD 1/5/9 (13820S, 1:1000), phospho-SMAD2/3 (8828S, 1:1000), SMAD1 (9743S, 1:1000), SMAD2/3 (3102S, 1:1000) and β-Tubulin (2146, 1:5000) were obtained from Cell Signaling; β-actin (sc-47778; 1:5000) were obtained from Santa Cruz Biotechnology. Appropriate secondary antibodies (anti-rabbit and anti-mouse) coupled to HRP were used (Dako).

Cell culture and reagents. Primary human pulmonary arterial endothelial cells (PAECs), human pulmonary arterial smooth muscle cells (PASMCs) were grown in basal medium EGM-2 and SmGM-2 supplemented with BulletKit (Lonza), respectively. All cells were cultured at 37° C. in 95% air and 5% CO₂. Experiments were performed at passages 5 to 10. Recombinant human IL-1β was purchased from Peprotech and used at concentrations of 10 ng/ml.

ELISA measurement for cell culture medium, serum, plasma, and lung tissue SCUBE1. Cell culture medium was collected at serial time points. Plasma or serum was derived from patient, mouse, or rat whole blood. The lung or myocardium tissue from human donor lung autopsy or from euthanized animals was homogenized with RIPA buffer with proteinase inhibitor. The medium, serum, plasma and tissue homogenate specimens were aliquoted and stored at −80° C. SCUBE1 levels were measured with human SCUBE1, rat and mouse SCUBE1 ELISA kits (OKEH01867, OKEI00879, and OKEH05018, respectively) purchased from Aviva Systems Biology, according to the manufacturer's instructions.

SCUBE1 and BMPR2 knockdown and lentiviral transduction of SCUBE1 transgene. PAECs were transfected with SCUBE1 and BMPR2 siRNA and Lipofectamine 2000 (Thermo Fisher Scientific). Non-targeted scrambled siRNA was used as control. The knockdown of target genes was confirmed with RT-qPCR.

Human SCUBE1 clone (SCUBE1-Bio-His, plasmid #53415) was purchased from Addgene. A 2.9 Kb SCUBE1 containing segment was cut and sub-cloned in the pCDH-CMV-MCS-EF1-copGFP (System Biosciences) using NotI/AscI restriction enzymes (New England Biolabs). The cloned plasmid was confirmed by DNA sequencing. HEK293T cells were co-transfected using Lipofectamine 2000 (Thermo Fisher Scientific) with lentiviral plasmids along with a packaging plasmid system (pPACK, System Biosciences), according to the manufacturer's instructions. Viral particles were harvested 48 hours after transfection, concentrated, sterile filtered (0.45 μm), and lentiviral titers were determined. Human PAECs were then infected at 60-70% confluence (16-24 hours incubation) with polybrene (8 μg/ml) for 2-3 days for gene transduction. The lentiviral parent vector expressing GFP was used as a control. The infection efficiency was assessed in each experiment by observing the GFP expression under a fluorescence microscope.

Cell exposure to hypoxia. Primary cells were exposed for 24-120 hours either to standard non-hypoxic cell-culture conditions, (20% O₂, 5% CO₂, with N₂ balance at 37° C.) or to hypoxia (0.2% O₂, 5% CO₂, with N₂ balance at 37° C.), in a modular hypoxia chamber, as previously described. Conditions were based on prior studies of human PAECs to allow for steady-state adaptation without non-specific cell death.

BrdU proliferation and caspase 3/7 apoptosis assays. All assays were performed per the manufacturers' instructions [BrdU Cell Proliferation Assay Kit (#6813, Cell Signaling); Caspase-Glo 3/7 Assay (Promega)]. For the caspase 3/7 assay, PAECs (10,000 cells/well) were incubated with Caspase-Glo 3/7 reagent in 96-well plate at room temperature for 1 hour, luminescence was recorded and normalized to protein content, as measured by BCA assay.

In vitro matrigel tube formation assay. Capillary tube formation was performed using a commercial kit (In vitro angiogenesis assay kit, Cultrex, #3470-096-K). Briefly, Matrigel with reduced growth factors was pipetted into pre-chilled 96-well plate (50 μl Matrigel per well) and polymerized for 30 min at 37° C. Following different treatments, HPAECs (2×10⁴ cells per well) were stained with Calcein AM (Cultrex, 1 μM) for 30 min at 37° C., resuspended in 100 μl of basic media, and seeded in Matrigel coated 96-well plate. After 4-6 h of incubation, tubular structures were photographed using an Olympus inverted microscope with a 20× magnification. The number of branch joint points was quantified by a blinded observer in triplicate determinations from 3 separate experiments.

PAH animal models. PAH rat models were generated in male Sprague-Dawley rats (10-14 week old, Charles River) injected with 60 mg/kg Monocrotaline (MCT), or injected with 20 mg/kg SU5416 (Sigma) followed by 3 weeks of normobaric hypoxia (10% 02) (Bertero T, 2014) and 2 weeks of normoxia. Prior to euthanasia, right heart catheterization was performed to confirm the elevated PAP. Thereafter, plasma and lung tissues were collected and stored in −80° C. for further studies.

PAH mouse model: As we recently reported (Bertero T, 2014), pulmonary inflammation resulting in severe PH in mouse was elicited in pulmonary interleukin-6 (IL-6) transgenic mice treated with hypoxia. C57BL/6 IL-6 transgenic male mice (10-12 weeks old) were subjected to 21 days of normobaric hypoxia (10% 02). Right heart catheterization was performed post-exposure, followed by tissue harvest. These rat and mouse procedures were approved by the Institutional Animal Care and Use Committee at the University of Pittsburgh (protocol number 16129515).

Experimental bacterial pneumonia mouse model. A Klebsiella pneumoniae (K. pneumoniae) bacterial pneumonia mouse model was generated as previously described (Olonisakin T F, 2016). Briefly, C57Bl/6J mice (JAX #000664) were anesthetized and 100 μL of K. pneumoniae bacterial slurry (strain 43816, serotype 2, American Type Culture Collection, Manassas, Va.) was administered intratracheally. Age and sex-matched mice were used for experiments. Forty-eight hours after K. pneumoniae inoculation, mice were euthanized, and blood and lung tissue were collected. All procedures were performed with approval of the Institutional Animal Care and Use Committee at the University of Pittsburgh (protocol number 18063096).

Acute myocardial infarction mouse model. Acute myocardial infarction in C57Bl/6J mice was induced by ligating the left coronary artery as previously described (Dutta P, 2012). Briefly, mice were anesthetized and intubated. Thoracotomy was performed and the pericardium was opened followed by permanent ligation of the left coronary artery at the site of the vessels' emergence past the tip of the left atrium. Myocardium histology was performed to delineate myocardial infarction. Sham-operated mice underwent the same procedure without coronary artery ligation. The plasma and left ventricular myocardium were collected on day 5 after surgery. All procedures were approved by the Institutional Animal Care and Use Committee at the University of Pittsburgh (protocol number 18083562).

Human subjects. For all the human subjects enrolled in study, informed consent was universally obtained, and all study procedures conformed to the ethical standards of the Declaration of Helsinki. All patients with known or suspected PH and referred for right heart catheterization (RHC) during their routine clinical care during 2014-2018 at two designated PH Comprehensive Care Centers—the University of Pittsburgh Medical Center (UPMC) and the Hospital of the University of Pennsylvania (HUP)—were eligible for inclusion. A total of 66 PH patients were from UPMC, including 52 Group 1 PAH patients and 14 Group 2 PH patients; 12 PH patients were from HUP, including 10 Group 1 PAH patients and 2 Group 2 PH patients. Procedures indicated for either diagnostic or disease monitoring purposes were included. Patients were prospectively enrolled by study staff before RHCs were performed. The study protocol was approved separately by the Institutional Review Boards (IRBs) from each respective institution (IRB No. STUDY19050364, University of Pittsburgh; IRB No. 818660, University of Pennsylvania). Non-PH control blood samples were collected from patients with RHC data confirming no PH (22 from UPMC and 9 from HUP), combined with 25 subjects from an established control cohort with no known pulmonary or cardiovascular diseases (IRB No. STUDY19070274).

Subjects with COPD were randomly selected from the Emphysema Research Registry in the University of Pittsburgh, each carrying a Forced Expiratory Volume to Forced Vital Capacity ratio, FEV1/FVC<0.7 and FEV1<80% predicted but Diffusing Capacity, DLCO>55% predicted. The study was approved by the Institutional Review Board for Human Subject Research at the University of Pittsburgh (IRB No. STUDY19120059).

Mechanically ventilated patients in the Medical or Cardiac Intensive Care Units at UPMC were enrolled in the University of Pittsburgh Acute Lung Injury and Biospecimen Repository from October 2011 to February 2020. For the present study, a subset of subjects (n=39) was selected from the cohort, meeting diagnostic criteria for the acute respiratory distress syndrome (ARDS) according to the Berlin criteria (ARDS Definition Task Force, 2012). Blood samples within 48 hours of intubation were collected from enrolled subjects. The study was approved by the University of Pittsburgh Institutional Review Board (IRB No. STUDY19050099).

Coronary artery disease (CAD) patients and age-, gender- and race-matched non-CAD controls were selected from an ongoing PCI Registry at UPMC. CAD was defined by coronary angiogram showing >50% stenosis requiring percutaneous coronary intervention. Control patients were defined with 0-49% stenosis on coronary angiography. The study was approved by the University of Pittsburgh Institutional Review Board (IRB No. STUDY990835).

Transthoracic echocardiography. The transthoracic echocardiogram (TTE) images of 36 PAH patients only from UPMC were reviewed by third-party clinician not involved directly in the clinical care. Only TTE studies performed within 3 months to the date of RHC and blood sample collection were analyzed. The measurement of RV dimensions, Tricuspid annular plane systolic excursion (TAPSE), and criteria for RV hypertrophy and dilation were based on standard protocol and American Society of Echocardiography consensus (Rudski L G, 2010).

Right heart catheterization. Clinically indicated right heart catheterizations were performed following a standard clinical protocol. A pulmonary artery (PA) catheter (Edwards, Irvine, Calif., USA) was advanced into the central venous system (superior vena cava [SVC]), right atrium (RA), right ventricle (RV), and PA by experienced operators in individuals at rest in the supine position after appropriate catheter calibration and zeroing. Pressure waveforms from the RA, PA, and pulmonary capillary wedge (PCW) positions were recorded in duplicate at end-expiration using the Xper Cardio Physiomonitoring System at UPMC (Philips, Melborne, Fla., USA) and Horizon Cardiology Cardiovascular Information System at HUP (McKesson, San Francisco, Calif., USA). Cardiac output (CO) was calculated by the Fick method using main PA and peripheral oxyhemoglobin saturations. Pulmonary vascular resistance was calculated based on the transpulmonary pressure gradient (mean PAP-PCWP) divided by CO.

Screening for World Symposium on Pulmonary Hypertension (WSPH) Group 1 PAH was first performed by evaluation of hemodynamics, namely via a mean pulmonary arterial pressure (mPAP)≥20 mmHg, a pulmonary capillary wedge pressure (PCWP)<15 mmHg, and a pulmonary vascular resistance (PVR)>3 Wood units (WU). These hemodynamic criteria followed the recently updated 2019 classification criteria (Simonneau G, 2019); notably, these samples predominantly fit the 2013 classification scheme as well (Simonneau G, 2013), since a vast majority of patient recruitment for blood samples was performed prior to 2018. After hemodynamic identification, third-party expert clinicians reviewed clinical notes and relevant studies for a determination of WSPH Pulmonary Hypertension Classification. Notably, a Group 1 PAH diagnosis was made only after excluding patients with confounding variables from etiologies more consistent with left heart disease, hypoxic lung disease, and chronic thromboembolism. Group 2 PH was defined by elevated mPAP with PCWP≥15 mmHg and known left heart disease, again as reviewed by third-party expert clinicians.

Transthoracic echocardiography. Transthoracic echocardiographic (TTE) images of 49 PAH patients from UPMC were analyzed by a third-party clinician not involved directly in each patient's clinical care. Only TTE studies performed within 3 months to the date of RHC and blood sample collection were analyzed. The measurement of RV dimensions, tricuspid annular plane systolic excursion (TAPSE), and criteria for RV hypertrophy and dilation were based on standard protocol and American Society of Echocardiography consensus (Rudski L G, 2010).

Blood sample, lung tissue, and heart tissue collection. Peripheral samples were drawn from human subjects described above (Human subjects). Samples were transferred into BD Vacutainer® tubes (BD, Franklin Lakes, N.J., USA), treated with standard anticoagulant ethylenediaminetetraacetic acid (EDTA), and subsequently spun at 2800 RCF in a Medilite Centrifuge (Thermo Scientific, Waltham, Mass., USA) for 10 min to initiate plasma separation.

Lung tissues from non-diseased normal controls, PAH and COPD patients as well as myocardial tissues from non-diseased controls, ischemic cardiomyopathy (ICM), and nonischemic cardiomyopathy (NICM) patients were collected from rapid lung biopsy or lung/heart transplant procedures, flash frozen, and stored at −80° C. at UPMC. These procedures were approved by the institutional review board (at UPMC (IRB No. PRO14010265 and CORID No. 722)).

Statistical analysis. All data are represented as mean±standard deviation (SD) or median with 25th and 75th (Q1-Q3) interquartile range, depending on data distribution. For cell culture data, these represent at least 3 independent experiments performed in triplicate. Normality of data distribution was confirmed by Shapiro Wilk testing. The categorical variables are presented in count and also percentage. For comparisons between two groups, a 2-tailed Student's t-test was used for normally distributed data. For comparisons among more than two normally distributed groups, one-way analysis of variance (ANOVA) testing was performed with post-hoc Bonferroni test. For non-normally distributed data, Mann-Whitney nonparametric test was performed for pairwise comparisons; Kruskal-Wallis test was performed with post-hoc Dunn's Multiple Comparison test for comparisons among non-normally distributed groups. A P-value less than 0.05 was considered significant. Non-parametric Spearman rank correlation was performed to determine the association of variables with calculated correlation coefficient (rho). ROC analysis was performed using the statistics package included with Graphpad Prism software (version 8.30). The Area Under Curve (C-statistics) was calculated using trapezoidal rule for empirical Sensitivity and 1-Specificity in any cut point (DeLong E R, 1988). The test sensitivity and specificity at various thresholds were calculated by the Clopper-Pearson method (Clopper C J, 1934). The optimal cut point in ROC curve was determined by Youden's test (Youden W J, 1950). A diagnostic odds ratio for the optimum cut point was calculated by dividing the positive likelihood ratio over the negative likelihood ratio, as previously reported (Glas A S, 2003).

### REFERENCES

1. Simonneau G, Montani D, Celermajer D S et al. Haemodynamic definitions and updated clinical classification of pulmonary hypertension. The European respiratory journal 2019; 53.
2. Rabinovitch M. Molecular pathogenesis of pulmonary arterial hypertension. The Journal of clinical investigation 2012; 122:4306-13.
3. Machado R D, Pauciulo M W, Thomson J R et al. BMPR2 haploinsufficiency as the inherited molecular mechanism for primary pulmonary hypertension. American journal of human genetics 2001; 68:92-102.
4. Farber H W, Miller D P, Poms A D et al. Five-Year outcomes of patients enrolled in the REVEAL Registry. Chest 2015; 148:1043-54.
5. Brown L M, Chen H, Halpern S et al. Delay in recognition of pulmonary arterial hypertension: factors identified from the REVEAL Registry. Chest 2011; 140:19-26.
6. Nikolic I, Yung L M, Yang P et al. Bone Morphogenetic Protein 9 Is a Mechanistic Biomarker of Portopulmonary Hypertension. American journal of respiratory and critical care medicine 2019; 199:891-902.
7. Anwar A, Ruffenach G, Mahajan A, Eghbali M, Umar S. Novel biomarkers for pulmonary arterial hypertension. Respiratory research 2016; 17:88.
8. de Jesus Perez V A, Alastalo T P, Wu J C et al. Bone morphogenetic protein 2 induces pulmonary angiogenesis via Wnt-beta-catenin and Wnt-RhoA-Rac1 pathways. The Journal of cell biology 2009; 184:83-99.
9. Teichert-Kuliszewska K, Kutryk M J, Kuliszewski M A et al. Bone morphogenetic protein receptor-2 signaling promotes pulmonary arterial endothelial cell survival: implications for loss-of-function mutations in the pathogenesis of pulmonary hypertension. Circulation research 2006; 98:209-17.
10. Gu M, Shao N Y, Sa S et al. Patient-Specific iPSC-Derived Endothelial Cells Uncover Pathways that Protect against Pulmonary Hypertension in BMPR2 Mutation Carriers. Cell stem cell 2017; 20:490-504 e5.
11. Tu C F, Yan Y T, Wu S Y et al. Domain and functional analysis of a novel platelet-endothelial cell surface protein, SCUBE1. The Journal of biological chemistry 2008; 283:12478-88.
12. Dai D F, Thajeb P, Tu C F et al. Plasma concentration of SCUBE1, a novel platelet protein, is elevated in patients with acute coronary syndrome and ischemic stroke. Journal of the American College of Cardiology 2008; 51:2173-80.
13. Wu M Y, Lin Y C, Liao W J et al. Inhibition of the plasma SCUBE1, a novel platelet adhesive protein, protects mice against thrombosis. Arteriosclerosis, thrombosis, and vascular biology 2014; 34:1390-8.
14. Turkmen S, Sahin A, Gunaydin M et al. The value of signal peptide-CUB-EGF domain-containing protein-1 (SCUBE1) in the diagnosis of pulmonary embolism: a preliminary study. Academic emergency medicine: official journal of the Society for Academic Emergency Medicine 2015; 22:922-6.
15. Patro R, Duggal G, Love M I, Irizarry R A, Kingsford C. Salmon provides fast and bias-aware quantification of transcript expression. Nature methods 2017; 14:417-419.
16. Soneson C, Love M I, Robinson M D. Differential analyses for RNA-seq: transcript-level estimates improve gene-level inferences. F1000Research 2015; 4:1521.
17. Love M I, Huber W, Anders S. Moderated estimation of fold change and dispersion for RNA-seq data with DESeq2. Genome biology 2014; 15:550.
18. Bertero T, Lu Y, Annis S et al. Systems-level regulation of microRNA networks by miR-130/301 promotes pulmonary hypertension. J Clin Invest 2014; 124:3514-28.
19. Yu Q, Tai Y Y, Tang Y, et al. BOLA (BolA Family Member 3) Deficiency Controls Endothelial Metabolism and Glycine Homeostasis in Pulmonary Hypertension. Circulation. 2019; 139(19):2238-2255.
20. Olonisakin T F, Li H, Xiong Z et al. CD36 Provides Host Protection Against Klebsiella pneumoniae Intrapulmonary Infection by Enhancing Lipopolysaccharide Responsiveness and Macrophage Phagocytosis. J Infect Dis 2016; 214:1865-1875.
21. Dutta P, Courties G, Wei Y et al. Myocardial infarction accelerates atherosclerosis. Nature 2012; 487:325-9.
22. ARDS Definition Task Force, Ranieri V M, Rubenfeld G D et al. Acute respiratory distress syndrome: the Berlin Definition. JAMA 2012; 307:2526-33.
23. Simonneau G, Gatzoulis M A, Adatia I et al. Updated clinical classification of pulmonary hypertension. Journal of the American College of Cardiology 2013; 62:D34-41.
24. Rudski L G, Lai W W, Afilalo J et al. Guidelines for the echocardiographic assessment of the right heart in adults: a report from the American Society of Echocardiography endorsed by the European Association of Echocardiography, a registered branch of the European Society of Cardiology, and the Canadian Society of Echocardiography. Journal of the American Society of Echocardiography: official publication of the American Society of Echocardiography 2010; 23:685-713.
25. DeLong E R, DeLong D M, Clarke-Pearson D L. Comparing the Areas under Two or More Correlated Receiver Operating Characteristic Curves: A Nonparametric Approach. Biometrics 1988; 44:837-45.
26. Clopper C J, Pearson E S. The Use of Confidence or Fiducial Limits Illustrated in the Case of the Binomial. Biometrika 1934; 26:404-413.
27. Youden W J. Index for rating diagnostic tests. Cancer 1950; 3:32-5.
28. Glas A S, Lijmer J G, Prins M H, Bonsel G J, Bossuyt P M. The diagnostic odds ratio: a single indicator of test performance. J Clin Epidemiol 2003; 56:1129-35.
29. Yuan H, Sehgal P B. MxA Is a Novel Regulator of Endosome-Associated Transcriptional Signaling by Bone Morphogenetic Proteins 4 and 9 (BMP4 and BMP9). PloS one 2016; 11:e0166382.
30. Yang R B, Ng C K, Wasserman S M et al. Identification of a novel family of cell-surface proteins expressed in human vascular endothelium. The Journal of biological chemistry 2002; 277:46364-73.
31. Sancisi V, Gandolfi G, Ragazzi M et al. Cadherin 6 is a new RUNX2 target in TGF-beta signalling pathway. PloS one 2013; 8:e75489.
32. Jung Y J, Isaacs J S, Lee S, Trepel J, Neckers L. IL-1beta-mediated up-regulation of HIF-1alpha via an NFkappaB/COX-2 pathway identifies HIF-1 as a critical link between inflammation and oncogenesis. FASEB journal: official publication of the Federation of American Societies for Experimental Biology 2003; 17:2115-7.
33. Machado R D, Eickelberg O, Elliott C G et al. Genetics and genomics of pulmonary arterial hypertension. Journal of the American College of Cardiology 2009; 54:532-42.
34. Johnson D W, Berg J N, Baldwin M A et al. Mutations in the activin receptor-like kinase 1 gene in hereditary haemorrhagic telangiectasia type 2. Nature genetics 1996; 13:189-95.
35. McAllister K A, Grogg K M, Johnson D W et al. Endoglin, a TGF-beta binding protein of endothelial cells, is the gene for hereditary haemorrhagic telangiectasia type 1. Nature genetics 1994; 8:345-51.
36. Shintani M, Yagi H, Nakayama T, Saji T, Matsuoka R. A new nonsense mutation of SMAD8 associated with pulmonary arterial hypertension. Journal of medical genetics 2009; 46:331-7.
37. Austin E D, Loyd J E. The genetics of pulmonary arterial hypertension. Circulation research 2014; 115:189-202.
38. Larkin E K, Newman J H, Austin E D et al. Longitudinal analysis casts doubt on the presence of genetic anticipation in heritable pulmonary arterial hypertension. American journal of respiratory and critical care medicine 2012; 186:892-6.
39. Love M I, Hogenesch J B, Irizarry R A. Modeling of RNA-seq fragment sequence bias reduces systematic errors in transcript abundance estimation. Nature biotechnology 2016; 34:1287-1291.
40. Wang R, Zhou S, Wu P et al. Identifying Involvement of H19-miR-675-3p-IGF1R and H19-miR-200a-PDCD4 in Treating Pulmonary Hypertension with Melatonin. Molecular therapy Nucleic acids 2018; 13:44-54.
41. Su H, Xu X, Yan C et al. LncRNA H19 promotes the proliferation of pulmonary artery smooth muscle cells through AT1R via sponging let-7b in monocrotaline-induced pulmonary arterial hypertension. Respiratory research 2018; 19:254.
42. Blasius A L, Giurisato E, Cella M, Schreiber R D, Shaw A S, Colonna M. Bone marrow stromal cell antigen 2 is a specific marker of type I IFN-producing cells in the naive mouse, but a promiscuous cell surface antigen following IFN stimulation. Journal of immunology 2006; 177:3260-5.
43. Bauer E M, Zheng H, Lotze M T, Bauer P M. Recombinant human interferon alpha 2b prevents and reverses experimental pulmonary hypertension. PloS one 2014; 9:e96720.
44. George P M, Oliver E, Dorfmuller P et al. Evidence for the involvement of type I interferon in pulmonary arterial hypertension. Circulation research 2014; 114:677-88.
45. Eggenberger J, Blanco-Melo D, Panis M, Brennand K J, tenOever BR. Type I interferon response impairs differentiation potential of pluripotent stem cells. Proceedings of the National Academy of Sciences of the United States of America 2019; 116:1384-1393.
46. Khan K A, Do F, Marineau A et al. Fine-Tuning of the RIG-I-Like Receptor/Interferon Regulatory Factor 3-Dependent Antiviral Innate Immune Response by the Glycogen Synthase Kinase 3/beta-Catenin Pathway. Molecular and cellular biology 2015; 35:3029-43.
47. Grimmond S, Larder R, Van Hateren N et al. Cloning, mapping, and expression analysis of a gene encoding a novel mammalian EGF-related protein (SCUBE1). Genomics 2000; 70:74-81.
48. Wang H, Ji R, Meng J et al. Functional changes in pulmonary arterial endothelial cells associated with BMPR2 mutations. PloS one 2014; 9:e106703.
49. Sa S, Gu M, Chappell J et al. Induced Pluripotent Stem Cell Model of Pulmonary Arterial Hypertension Reveals Novel Gene Expression and Patient Specificity. American journal of respiratory and critical care medicine 2017; 195:930-941.
50. Tsao K C, Tu C F, Lee S J, Yang R B. Zebrafish scube1 (signal peptide-CUB (complement protein C1r/C1s, Uegf, and Bmp1)-EGF (epidermal growth factor) domain-containing protein 1) is involved in primitive hematopoiesis. The Journal of biological chemistry 2013; 288:5017-26.
51. Rol N, Kurakula K B, Happe C, Bogaard H J, Goumans M J. TGF-beta and BMPR2 Signaling in PAH: Two Black Sheep in One Family. International journal of molecular sciences 2018; 19.
52. Kimura H, Weisz A, Ogura T et al. Identification of hypoxia-inducible factor 1 ancillary sequence and its function in vascular endothelial growth factor gene induction by hypoxia and nitric oxide. The Journal of biological chemistry 2001; 276:2292-8.
53. Tu C F, Su Y H, Huang Y N et al. Localization and characterization of a novel secreted protein SCUBE1 in human platelets. Cardiovascular research 2006; 71:486-95.
54. D'Alonzo G E, Barst R J, Ayres S M et al. Survival in patients with primary pulmonary hypertension. Results from a national prospective registry. Annals of internal medicine 1991; 115:343-9.
55. Benza R L, Miller D P, Barst R J, Badesch D B, Frost A E, McGoon M D. An evaluation of long-term survival from time of diagnosis in pulmonary arterial hypertension from the REVEAL Registry. Chest 2012; 142:448-456.
56. Malhotra R, Paskin-Flerlage S, Zamanian R T et al. Circulating angiogenic modulatory factors predict survival and functional class in pulmonary arterial hypertension. Pulmonary circulation 2013; 3:369-80.
57. Maron B A, Ryan J. J. A Concerning Trend for Patients With Pulmonary Hypertension in the Era of Evidence-Based Medicine. Circulation 2019; 139:1861-1864.
58. Miller W L, Grill D E, Borlaug B A. Clinical features, hemodynamics, and outcomes of pulmonary hypertension due to chronic heart failure with reduced ejection fraction: Pulmonary hypertension and heart failure. JACC. Heart failure. 2013; 1:290-299
59. Costard-Jackle A, Fowler M B. Influence of preoperative pulmonary artery pressure on mortality after heart transplantation: Testing of potential reversibility of pulmonary hypertension with nitroprusside is useful in defining a high risk group. Journal of the American College of Cardiology. 1992; 19:48-54
60. Wright S P, Moayedi Y, Foroutan F, Agarwal S, Paradero G, Alba A C, Baumwol J, Mak S. Diastolic pressure difference to classify pulmonary hypertension in the assessment of heart transplant candidates. Circulation. Heart failure. 2017; 10
61. Gerges C, Gerges M, Lang M B, Zhang Y, Jakowitsch J, Probst P, Maurer G, Lang I M. Diastolic pulmonary vascular pressure gradient: A predictor of prognosis in “out-of-proportion” pulmonary hypertension. Chest. 2013; 143:758-766
62. Papani R, Duarte A G, Lin Y L, Kuo Y F, Sharma G. Pulmonary arterial hypertension associated with interferon therapy: A population-based study. Multidisciplinary respiratory medicine. 2017; 12:1
63. Mizuno K, Nakane A, Nishio H, Moritoki Y, Kamisawa H, Kurokawa S, Kato T, Ando R, Maruyama T, Yasui T, Hayashi Y. Involvement of the bone morphogenic protein/SMAD signaling pathway in the etiology of congenital anomalies of the kidney and urinary tract accompanied by cryptorchidism. BMC Urol. 2017; 17: 112.

1. A method of treating a pulmonary hypertension in a subject comprising administering to the subject a therapeutically effective amount of a vector, wherein the vector comprises a polynucleotide that encodes SCUBE1 or a functional fragment thereof, the administration resulting in a reduction of a pulmonary arterial hypertension and/or a pulmonary vascular resistance in the subject.

2. The method of claim 1, wherein the subject has a pulmonary arterial hypertension (PAH) prior to treatment.

3. The method of claim 1, wherein the subject has a pulmonary vascular hypertension (PVH) with a high pulmonary vascular resistance prior to treatment.

4. The method of claim 1, wherein the polynucleotide is a DNA or a RNA.

5. The method of claim 1, wherein the vector is a viral vector.

6. The method of claim 5, wherein the viral vector is a lentiviral vector.

7. The method of claim 1, wherein the administration of the vector increases an amount of SCUBE1 in an endothelial cell.

8. The method of claim 7, wherein the endothelial cell is a pulmonary vascular endothelial cell.

9. The method of claim 7, wherein the endothelial cell is a pulmonary arterial endothelial cell.

10. The method of claim 7, wherein the administration of the vector increases angiogenesis in the endothelial cell as compared to a control.

11. The method of claim 7, wherein the administration of the vector increases proliferation of the endothelial cell as compared to a control.

12. The method of claim 7, wherein the administration of the vector decreases death of the endothelial cell as compared to a control.

13. The method of claim 7, wherein a phosphorylation of a Smad1/5/9 polypeptide is modified in the endothelial cell as compared to a control.

14. The method of claim 1, wherein the administration of the vector decreases a level of pulmonary arterial pressure in the subject.

15. The method of claim 1, wherein the administration of the vector decreases a level of pulmonary vascular resistance in the subject.

16. The method of claim 1, wherein the subject has a mutation in a gene encoding BMPR2.

17. The method of claim 1, further comprising identifying the subject as having a reduction of SCUBE1 in a plasma sample and/or a lung tissue sample relative to a control.

18. A method of treating a pulmonary hypertension in a subject comprising administering to the subject a therapeutically amount of a polypeptide, wherein the polypeptide comprises SCUBE1 or a functional fragment thereof, the administration resulting in a reduction of a pulmonary arterial hypertension and/or a pulmonary vascular resistance in the subject.

19. The method of claim 18, wherein the subject has a pulmonary arterial hypertension (PAH) prior to treatment.

20. The method of claim 18, wherein the subject has a pulmonary vascular hypertension (PVH) with a high pulmonary vascular resistance prior to treatment.

21. The method of claim 18, wherein the administration of the polypeptide increases angiogenesis in an endothelial cell as compared to a control.

22. The method of claim 21, wherein the administration of the polypeptide increases proliferation of an endothelial cell as compared to a control.

23. The method of claim 21, wherein the administration of the polypeptide decreases death of an endothelial cell as compared to a control.

24. The method of claim 21, wherein the endothelial cell is a pulmonary vascular endothelial cell.

25. The method of claim 24, wherein the pulmonary vascular endothelial cell is a pulmonary arterial endothelial cell.

26. The method of claim 21, wherein the administration of the polypeptide decreases a level of pulmonary arterial pressure in the subject.

27. The method of claim 21, wherein the administration of the polypeptide decreases a level of pulmonary vascular resistance in the subject.

28. The method of claim 21, wherein the subject has a mutation in a gene encoding BMPR2.

29. The method of claim 21, further comprising identifying the subject as having a reduction of SCUBE1 in a plasma sample and/or a lung tissue sample derived from the subject relative to a control.

30. A method of diagnosing or prognosing a pulmonary arterial hypertension in a subject comprising detecting a reduction of a SCUBE1 polynucleotide or polypeptide in a biological sample derived from the subject as compared to a control, and diagnosing or prognosing the subject with the pulmonary arterial hypertension following the detection of the reduction of SCUBE1.

31. The method of claim 30, wherein the biological sample is a plasma sample or a lung tissue sample and a reduction of the SCUBE1 polypeptide is detected.

32. The method of claim 30, wherein the subject has an increased level of pulmonary arterial pressure relative to a control.

33. The method of claim 30, wherein the subject has an increased level of pulmonary vascular resistance relative to a control.

34. The method of claim 30, wherein a degree of the reduction positively correlates with severity of the pulmonary arterial hypertension.

35. The method of claim 30, wherein the subject has a mutation in a gene encoding BMPR2.

36. The method of claim 30, further comprising administering to the subject a therapeutically effective amount of a vector, wherein the vector comprises a polynucleotide that encodes SCUBE1 or a functional fragment thereof.

37. The method of claim 30, further comprising administering to the subject a therapeutically effective amount of a polypeptide, wherein the polypeptide comprises SCUBE1 or a functional fragment thereof.

38. The method of claim 1, wherein the subject is a human.


SEQUENCE LISTING (4 sequences)

SEQ ID NO: 1 - protein, 363 aa, Homo sapiens
MGAAAVRWHLCVLLALGTRGRLAGGSGLPGSVDVDECSEGTDDCHIDAICQNTPKSYKCLCKPGYKGEGKQCEDIDECENDYYNGGCVHECINIPGNYRCTCFDGFMLAHDGHNCLDVDECQDNNGGCQQICVNAMGSYECQCHSGFFLSDNQHTCIHRSNEGMNCMNKDHGCAHICRETPKGGVACDCRPGFDLAQNQKDCTLTCNYGNGGCQHSCEDTDTGPMCGCHQKYALHSDGRTCIETCAVNNGGCDRTCKDTATGVRCSCPVGFTLQPDGKTCKDINECLVNNGGCDHFCRNTVGSFECGCRKGYKLLTDERTCQDIDECSFERTCDHICINSPGSFQCLIKLLRSKVSRFLRPYK

SEQ ID NO: 2 - DNA, 9794 bp, Homo sapiens
  mRNA (1)..(9794)
agcccgagcggtggcggagcggcgagcagcgagcagcgcctgcgggagcggccggtcggtgggtccccgcgccccgcacgcccgcacgcccagcggggcccgcattgagcatgggcgcggcggccgtgcgctggcacttgtgcgtgctgctggccctgggcacacgcgggcggctggccgggggcagcgggctcccagggtcagtcgacgtggatgagtgctcagagggcacagatgactgccacatcgatgccatctgtcagaacacgcccaagtcctacaaatgcctctgcaagccaggctacaagggggaaggcaagcagtgtgaagacattgacgagtgtgagaatgactactacaatgggggctgtgtccacgagtgcatcaacatcccggggaactacaggtgtacctgctttgatggcttcatgctggcacacgatggacacaactgcctggatgtggacgagtgtcaggacaataatggtggctgccagcagatctgcgtcaatgccatgggcagctacgagtgtcagtgccacagtggcttcttccttagtgacaaccagcatacctgcatccaccgctccaatgagggtatgaactgcatgaacaaagaccatggctgtgcccacatctgccgggagacgcccaaaggtggggtggcctgcgactgcaggcccggctttgaccttgcccaaaaccagaaggactgcacactaacctgtaattatggaaacggaggctgccagcacagctgtgaggacacagacacaggccccacgtgtggttgccaccagaagtacgccctccactcagacggtcgcacgtgcatcgagacgtgcgcagtcaataacggaggctgcgaccggacatgcaaggacacagccactggcgtgcgatgcagctgccccgttggattcacactgcagccggacgggaagacatgcaaagacatcaacgagtgcctggtcaacaacggaggctgcgaccacttctgccgcaacaccgtgggcagcttcgagtgcggctgccggaagggctacaagctgctcaccgacgagcgcacctgccaggacatcgacgagtgctccttcgagcggacctgtgaccacatctgcatcaactccccgggcagcttccagtgcctgtgtcaccgcggctacatcctctacgggacaacccactgcggagatgtggacgagtgcagcatgagcaacgggagctgtgaccagggctgcgtcaacaccaagggcagctacgagtgcgtctgtcccccggggaggcggctccactggaacgggaaggattgcgtggagacaggcaagtgtctttctcgcgccaagacctccccccgggcccagctgtcctgcagcaaggcaggcggtgtggagagctgcttcctttcctgcccggctcacacactcttcgtgccagactcggaaaatagctacgtcctgagctgcggagttccagggccgcagggcaaggcgctgcagaaacgcaacggcaccagctctggcctcgggcccagctgctcagatgcccccaccacccccatcaaacagaaggcccgcttcaagatccgagatgccaagtgccacctccggccccacagccaggcacgagcaaaggagaccgccaggcagccgctgctggaccactgccatgtgactttcgtgaccctcaagtgtgactcctccaagaagaggcgccgtggccgcaagtccccatccaaggaggtgtcccacatcacagcagagtttgagatcgagacaaagatggaagaggcctcagacacatgcgaagcggactgcttgcggaagcgagcagaacagagcctgcaggccgccatcaagaccctgcgcaagtccatcggccggcagcagttctatgtccaggtctcaggcactgagtacgaggtagcccagaggccagccaaggcgctggaggggcagggggcatgtggcgcaggccaggtgctacaggacagcaaatgcgttgcctgtgggcctggcacccacttcggtggtgagctcggccagtgtgtgtcatgtatgccaggaacataccaggacatggaaggccagctcagttgcacaccgtgccccagcagcgacgggcttggtctgcctggtgcccgcaacgtgtcggaatgtggaggccagtgttctccaggcttcttctcggccgatggcttcaagccctgccaggcctgccccgtgggcacgtaccagcctgagcccgggcgcaccggctgcttcccctgtggagggggtttgctcaccaaacacgaaggcaccacctccttccaggactgcgaggctaaagtgcactgctcccccggccaccactacaacaccaccacccaccgctgcatccgctgccccgtcggcacctaccagcccgagtttggccagaaccactgcatcacctgtccgggcaacaccagcacagacttcgatggctccaccaacgtcacacactgcaaaaaccagcactgcggcggcgagcttggtgactacaccggctacatcgagtcccccaactaccctggcgactacccagccaacgctgaatgcgtctggcacatcgcgcctcccccaaagcgcaggatcctcatcgtggtccctgagatcttcctgcccatcgaggatgagtgcggcgatgttctggtcatgaggaagagtgcctctcccacgtccatcaccacctatgagacctgccagacctacgagaggcccatcgccttcacctcccgctcccgcaagctctggatccagttcaaatccaatgaaggcaacagcggcaaaggcttccaagtgccctatgtcacctacgatgaggactaccagcaactcatagaggacatcgtgcgcgatgggcgcctgtacgcctcggagaaccaccaggaaattttgaaagacaagaagctgatcaaggccctcttcgacgtgctggcgcatccccagaactacttcaagtacacagcccaggaatccaaggagatgttcccacggtccttcatcaaactgctgcgctccaaagtgtctcggttcctgcggccctacaaataaccggggggagcggccctgcctgggggtggcctggtccgcggagggtgcacctgccctccacagtgggagctgcatgggcctccacaccaccttgggaaccccatggcactgcccttcagggaagccgaccagcccatggagaccgagcccaggcacccttcggacccgctgcccctgtgggagcaccctgcttcaggaagcctccctccctccctctgcctcccttccccaggacaccaagagcgccctctcctgagccctggcagaccgactgcaggtagcaggattgcaggaccctctgcctggcctggcgtttcaggagagaggggaagtggggcctgtgctctgggaggcgtggtcatccgagacaggagtccaggggagagaggaggggacaaaggcgccgtctgggggaggtcgatgagcctgtgctggcatccgcgggccccacgctttgccaactcctccagccacaggcaaggccacggctccgggctgttgcgctctaagggttctgtgattggatggaacagagctgctggggaggagactggaagtttctgcattccttcaacagaacatttaatgaagtactctatatatatatataaatatatatataaatatatatatatacttctatttgtgggtactttaggaaaatgccctttggtcactgtaaatatgaattgtgaccccatcccttcccgcatgagcccagtgagtcccagcagctatcagcctccctgaacgattaaacagctcctcccagcatttgcatttgcccttctgtttctgtgaggccggcagcccttggggctggggagagcacagcttgccttggacttgcttctgaacacgaattcttcgagaactaggctcagatgctctgctggagtgttggcctcaccccagtccccttgccagcaacatcactgcccttggacgagactcagtggaagacacttcccctcggacccgttcccaggaggtgcaggggcccagcaataaacggcgaaaggggccttggctcttgccctgtggcagtgaccttggggtccgtactatgcccaggtgtcagcaggaaacaaggccttgctcacaggagcacaggctgtaccaccacaaggagcctcagagcggccagtgaggaaaggggagagagcagagcttgctgtcagaggccaggctcccagtgcagcctcctcttgctacgccatgccacccccattcacagggaaacctctttccttcctataaaggaaagaggttgaattgactcacatttccacatggcttgggaggcctcaggaaacttacaatcatggcggagggtgaaggggaagcaagcgccttcttcacaaggcagcaggagagagaagcaagcagggaaatgtcgggagaaagagagtctgggctgtgaccgagtgcatgtgtcccacccagccggaaggccagcgcctgggatggagccagcagaaccccatgccgaggtccaggaggccttgcctgctcttcatctgcctgcgagctgtaggacctgcatgggaaatggtctgtgtgtttcaggttaacaaatactgaaaatcccaaggaacctgggagaatctgaaaatgaactccagtcttcagctcgaagttgcctctagggttcctcctggtcacccccgggcccagcctggctggcgagaaacaaacacggtttgagaggctgtggcacactgaggaccacacagatggaggcagccaagtgaggtccctggaggttcagggcttggtgacgaggtaggccgtgatcacatattctacagtccaaaagagggcccggagctcagcaattactcacgccgtcacatgtgcccacagtagcgggctccgcggcgtggctcctgctgccatcagctgccttctctgctgaagtccaactcaagtcggagagagagtggagcctctgcacttggggagtaggtcagtgtcaactaacaagccctgccaggggccactgccgctcctctggtcacagtccacgcaggccagccctggtttctggaaagaggcctccctccctccctttgtggagccgtcagtcatccccaagatgagaggcgagcatcctgccgagtgagcctgcagcaacagctcctgaggaaggtgccgggaggggtgtcgcctcttcacgagtgttctccagagaatcagaacccagaggatacagacgtagatgtgtgaggggctgcattatgggaactggctcacagattctggaggccaacaagtcccacgatcggctgtctgcgattgggagaccaggagaagccacaccgtaactcagtctgagtccaaagcccgagaagccaggggccaccctgtgagtcccagcatccaaagccccgagaacgaggaagatggatgtcccagctcccaaagggaggattctcccttcctcctccttcttttctattggggccctcaacagattggatggagtggatgtcttcactcagtctgccaattcccatcctaacctcttctggaaacaccctagcatgttttcccagctagctgggtatccgttcacccaaccaagacgaccatgcaatcaaccatcacacttaaatgtttttgttttgttttttttgttttgttttttgttttctttttttgttttcttttttttttttttttttttttttgagatggagtctcgctctgtcacccaggctggagtgcagtggcgcgatcttggttcactgcaagctccgcctcccgggttcacgccattctcctgcctcagcctcccgagtagctgggactacaggcacccaccaccttgcccggctaatttttttgtatttttagtagagacagggtttcaccatattagccaggatggtctcgatctcctgacctcgtgatccgcccacctcggcctcccaaagtgctgggattacaggcgtgagccaccacgcccagccagggttttcttttttaatcctttccagaaacagaaagagctcttaacctctctgagctctttctgcaattcctttgggcgatttctgaaagttgccacccacgtactcacagatccactgcccgcctctcctcccctcatgttccaggactcaccaccgtggtcagaccatgcccagtgtgcccagggcaaagttggatgggcaggtgcagacgaggcccagccttcatcctgctttccctcgcaagcccttctatagctcccagcccacgggagaggccatgaggagcgcgaggtcgccagcagccccactgggcctcccacaccatctgagtgacttgaattccagggttgggctctgtcttgagtctccacatcagtctcacaccagggaacttgggggtcccccgttaaactacagctgaccctgggcctcagggcgggatggtcccaaggtgagctccgtggacagcaaagcgcatttcttccagttggcatgttgtcgaagccaatggcagcttgactgatgtgaggtggctattccacctggttcaagatggaggtgtagagaaaagggaagtggggaacaaggactgggccaggtaggacggatccacgtaacgaggcccaagaaaaaatggaagctgggggcagagagtccaggaggcccccatgctgtctgaggggaggccggcatagcccctcctcgactgtcgcctgagcagctgatgaggtccccagcgccgcacgctgtgctccagggagaggagccaagaagcaagcgggctgctgggagtccctgcactgccatctggccgggacggaggcgcctgcccggtctccaggctcctctgagcttgggtcagctcctaatggagcatggcaccccagggaaccagaggaactctgtgaagaaaggcatggaaaaccggggtgtctcccgcacagcttcccaggagtcaggtgggacccaggcgtggggggacctttctgccccacattcctggcattccccccaagtttatttctactttccagaaacaagccacgggaagggaaggaacctgcagcagggtttcgaagacagccaggccctccgtgattgagggctcatatagccgctgagggcagcctgcagccaggtggcctcacctgctgcccagaattaggctgtgccttttcccagtccttccccatgccagagcctctccccatctttccccgcgccagagcctcacagccacagccacaccaacctccctcctctcaagcagcagaatcagcctacgctccccagcctcagtgcccctgccccacacaggtcacgctggcaaggcgatgaccagtttccgaaacgctggatccccaagggtacagtgtggcccagtcctcccccacctccatggtggcccaggctgcagggctcacggtgggcccacggtggaggcctcaggcagctttctctccagctacccattggcttaggacacacagataaagtagagctgaaacacgggccaaaacatccccagacaaggatggcacaagaggccaaatttgaaggccttgggagagaagccatttcccctacctctaacgagcgtagttttctgtctgaatggccccccttttcatgcacgctcgttcttaagagacgcaccatccgcaggcagcgtctcagtgaagcctgtcccctgggcttgtccacggcaggaggcccctggcctgtttaagccataccgatgcttaatttacatttttggagttgaatattattccggtgcctccaccacccccagcagaaaaaataagatactgaaataagttgagtcatttcgcaaacatttactgagcacctcctgtgtgccaggctcctagaaagacagggagttggtgccctgcacagatgacaaactgccccagacagcagacagacaaacaaactgccccggacagcagacagacaaacagacacattgccctggacagcagagagacaaacagacacactgccccggacagcagacagacagacacgttgccccggacagcagacagaaaaactgccccggacagcagacagacagacaaactgccccagacagcagacagacaaactgccccggaaagcagacagcaaactgccccagacagcagacagacagacacacttccccagacagcagatagacagacaccctgccccagacagcagacaagctgccctggacagcctgctcaccttgcctgccacagtgcatgccccacagggagaagccttccttctcacccagggagtccagccattttcacctcccctggtccatgctcaggaagtctggtcaccctggcaaactgcacctgacattgaggtcccttttcaccaaagatgcacaaggtgtccatgccgagccgacatcattcaggccaagggcagaggatgtaatgaggggtggagtgaggatgtggagagagagcagagagcgcctcctattctaccgcggctgcctctgcccccacagcacccacagatccctggcccttccaggagtaagtcctgggcaggcagagctggggttggcctgatcccacctgccttgctcttggaaaggacatcaggtgtgggcaaagcatccttgaagcccagacaacacaggcatggcctgcgtgccctcccagcatctgctgcgatggcctcaaggcctctccagtcccctgctgcaccatgccactcagagcccctgagtgagccccctggaattgtctgcaccccccaagccccaacaggaagaagggaaaggtggggaagctgacgctcactcagtgcccactgggtgccaggctctgggctcaaccctctaatatccacacattccatccccccagccccatgtagatggtggggttaagaccattcttcagatgtggaaatgaaggctctgggagaccacaggacatgcccaaggcaaagagttggtagtccctggatttgggaatcaaactcaagcccatctaatccaagggtaggaaatgtctctaaaacgttgatcaatctgaagcttacagattggaaactgggccaggtcagccctggcggtggagtccagagacagagagaaggctgcagggaggggagagaacaggaggatcccgggccagggtgacttcagtgacattccttccctccttcacaagcacgaaaaactcgttcggagaaaagacagcttctgccttccctaccaggcggacagggtactcactgtgccgacgtgagcagacacagccagaggctcaagtgcccttgccgagtgtccccactgctcttggggaggccccggcacagccctgcagagatgcaagtaagagagtgcccagtgccctcggcagatgggtggcagaaagcaggaggaggaagagggagctgaccgatgccgggtgtcttactccgctgagatcagaacctccaacatgccgagcagacgcaggccacattgctcggggaacaggacaccagggcgctgtgctagcggcccgcagacttcacttctggctcctgtgagggcactgtattgtccagacgcttggcaacgactctgcctgcaagtggcaaataccaacataaccacatcttccttttagttgccacaaatattcagttttgttttatccatcatgtcccccatatgcatgtaatgaaacatttgtgaaaatcactttaataataaacaggaaatgaacctaaa

SEQ ID NO: 3 - protein, 988 aa, Homo sapiens
MGAAAVRWHLCVLLALGTRGRLAGGSGLPGSVDVDECSEGTDDCHIDAICQNTPKSYKCLCKPGYKGEGKQCEDIDECENDYYNGGCVHECINIPGNYRCTCFDGFMLAHDGHNCLDVDECQDNNGGCQQICVNAMGSYECQCHSGFFLSDNQHTCIHRSNEGMNCMNKDHGCAHICRETPKGGVACDCRPGFDLAQNQKDCTLTCNYGNGGCQHSCEDTDTGPTCGCHQKYALHSDGRTCIETCAVNNGGCDRTCKDTATGVRCSCPVGFTLQPDGKTCKDINECLVNNGGCDHFCRNTVGSFECGCRKGYKLLTDERTCQDIDECSFERTCDHICINSPGSFQCLCHRGYILYGTTHCGDVDECSMSNGSCDQGCVNTKGSYECVCPPGRRLHWNGKDCVETGKCLSRAKTSPRAQLSCSKAGGVESCFLSCPAHTLFVPDSENSYVLSCGVPGPQGKALQKRNGTSSGLGPSCSDAPTTPIKQKARFKIRDAKCHLRPHSQARAKETARQPLLDHCHVTFVTLKCDSSKKRRRGRKSPSKEVSHITAEFEIETKMEEASDTCEADCLRKRAEQSLQAAIKTLRKSIGRQQFYVQVSGTEYEVAQRPAKALEGQGACGAGQVLQDSKCVACGPGTHFGGELGQCVSCMPGTYQDMEGQLSCTPCPSSDGLGLPGARNVSECGGQCSPGFFSADGFKPCQACPVGTYQPEPGRTGCFPCGGGLLTKHEGTTSFQDCEAKVHCSPGHHYNTTTHRCIRCPVGTYQPEFGQNHCITCPGNTSTDFDGSTNVTHCKNQHCGGELGDYTGYIESPNYPGDYPANAECVWHIAPPPKRRILIVVPEIFLPIEDECGDVLVMRKSASPTSITTYETCQTYERPIAFTSRSRKLWIQFKSNEGNSGKGFQVPYVTYDEDYQQLIEDIVRDGRLYASENHQEILKDKKLIKALFDVLAHPQNYFKYTAQESKEMFPRSFIKLLRSKVSRFLRPYK

SEQ ID NO: 4 - protein, 371 aa, Homo sapiens
MIFPWKCQSTQRDLWNIFKLWGWTMLCCDFLAHHGTDCWTYHYSEKPMNWQRARRFCRDNYTDLVAIQNKAEIEYLEKTLPFSRSYYWIGIRKIGGIWTWVGTNKSLTEEAENWGDGEPNNKKNKEDCVEIYIKRNKDAGKWNDDACHKLKAALCYTASCQPWSCSGHGECVEIINNYTCNCDVGYYGPQCQFVIQCEPLEAPELGTMDCTHPLGNFSFSSQCAFSCSEGTNLTGIEETTCGPFGNWSSPEPTCQVIQCEPLSAPDLGIMNCSHPLASFSFTSACTFICSEGTELIGKKKTICESSGIWSNPSPICQKLKSFSMIKEGDYNPLFIPVAVMVTAFSGLAFIIWLARRLKKGKKSKRSMNDPY